(19) United States
(12) Patent Application Publication
(10) Pub. No.: US 2024/0352060 A1
(43) Pub. Date: Oct. 24, 2024

(54) METHODS FOR THE SYNTHESIS OF PEPTOIDS USING GREEN SOLVENTS

(71) Applicant: The Governing Council of the University of Toronto, Toronto (CA)

(72) Inventors: Helen Tran, Toronto (CA); Abigail Mae Clapperton, Markham (CA)

(21) Appl. No.: 18/620,645

(22) Filed: Mar. 28, 2024

(30) Foreign Application Priority Data
Mar. 29, 2023 (CA) 3194505

(51) Int. Cl. C07K 1/04; C07K 1/06

(52) U.S. Cl. CPC C07K 1/042; C07K 1/064

(57) ABSTRACT

Disclosed herein are methods for making a poptoid using green solvents such as ethyl acetate, DMSO and/or mixtures thereof. Methods using an acylation solvent that is DMSO or EIOAc or mixtures thereof as well as methods using an acylation wash solvent comprising DMSO wherein the DMSO is greater than 30% v/v are described.

### FIELD

The present application claims the benefit of priority of Canadian patent application no. 3, 194,505 filed on Mar. 29, 2023 the contents of which are incorporated herein by reference.

The present disclosure relates to methods of synthesizing peptoids using alternative solvents.

### INTRODUCTION

Peptoids, or N-substituted glycine polymers, are an important platform for the development of new materials for therapeutic, cryopreservation, and biosensing applications.¹⁻¹⁰ The properties of these materials can be easily tuned with the diverse array of side chains able to be incorporated into well-defined sequences. This high level of sequence control is possible with the submonomer solid-phase synthesis strategy (FIG. 1A), which relies on iterative acylation and displacement reactions to grow the peptoid chain. 11 The versatility of this method lies in the ability to incorporate any commercially or synthetically available amine into peptoids with chain lengths up to 50 units.¹² This solid-phase protocol has been widely adopted among peptoid researchers and optimized for unique side chains, specific backbone geometries, and adaptation onto automated synthesizers.¹³⁻¹⁸

Despite the advancements made in improving the versatility of the submonomer solid-phase method, limitations in the sustainability and safety of these protocols remain. The synthesis of peptoids is reliant on hazardous solvents, N,N-dimethylformamide (DMF) and N-methyl-2-pyrrolidone (NMP), used in large quantities for resin swelling, washing, and solubilizing the required chemical reagents (FIG. 1A).^{11,12,19,20} With 2 steps required for each monomer addition and repetitive washings following each reaction, there is significant hazardous solvent consumption and waste generation in solid-phase peptoid synthesis. For example, 225 mL of DMF and NMP is required in the 62 μmol scale synthesis of a peptoid with 6 monomer units and 630 mL is required for the addition of 18 monomer units (FIG. 1B). 12 While DMF and NMP have shown excellent performance as solvents for peptoid synthesis in both academic and industrial research environments, their use has been restricted by the European Union due to their reprotoxicity.²¹⁻²⁷

These restrictions motivate the search for alternative solvents with improved safety profiles that can maintain the efficiency of solid-phase peptoid synthesis.

### SUMMARY

Peptoids are a class of sequence-controlled polymers that provide a tunable platform for the design of bioinspired materials. Solid-phase synthetic methods offer control over the polypeptoid sequence and have been optimized to improve reaction efficiency and versatility. However, these solid-phase strategies rely on the use of toxic solvents, such as N,N-dimethylformamide (DMF) and N-methyl-2-pyrrolidone (NMP), resulting in significant hazardous solvent consumption and waste generation. The solid-phase synthesis of peptoids with elimination of such solvents as DMF and NMP and their replacement with green solvents is described which may for example minimize the environmental impact and improve the sustainability of peptoid synthesis. As demonstrated herein, the swelling performance of the green solvents is investigated. Further it is shown that the purity profile and yield of the final peptoids are not adversely affected when compared to those synthesized in traditional solid-phase solvents. Furthermore, the greener methods are adapted for use on automated synthesizers. The synthesis of peptoids with different sequences and longer chain lengths is also demonstrated.

An aspect of the invention includes a method of synthesizing a peptoid, the method comprising
a) reacting a solid support, optionally a solid support resin, comprising a terminal amine group, with a haloacetic acid (HA) in the presence of a HA activating agent in an acylation solvent, to obtain a solid support resin comprising an acylated amino group;
b) washing the solid support, optionally a solid support resin, comprising the acylated amino group with an acylation wash solvent to obtain a washed solid support resin comprising the acylated amino group;
c) reacting the washed solid support, optionally a solid support resin, comprising the acylated amino group with a primary amine in a displacement solvent, to obtain a washed solid support, optionally a solid support resin, comprising an acylated resin-bound amide;
d) washing the solid support, optionally a solid support resin, comprising the acylated resin-bound amide, with a displacement wash solvent, to obtain an attached peptoid monomer comprising an amine group;
e) repeating steps a)-d) at least once, where the amine group is the terminal amine group in step a) to obtain a peptoid;
wherein the acylation solvent, and the displacement solvent, are polar aprotic solvents that are non-reactive to secondary amines and optionally the acylation solvent provides for a resin swelling of at least 1.25, 1.3, 1.35, 1.4, 1.45 or 1.5 and/or the acylation wash solvent is able to solubilize 1,3-diisopropylurea; or
wherein the acylation solvent is selected from EtOAc, or a DMSO and EtOAc mixture, or
wherein the acylation wash solvent is selected from DMSO, EtOAc, or combinations thereof, and
wherein when the acylation solvent is EtOAc, the acylation wash solvent is not EtOAc, and when the acylation wash solvent is DMSO, the acylation solvent is not DMSO.

In an embodiment, the haloacetic acid is selected from a bromoacetic acid, chloroacetic acid, or iodoacetic acid.

In an embodiment, the haloacetic acid is bromoacetic acid.

In an embodiment, the acylation solvent is or comprises EtOAc.

In an embodiment, the acylation solvent is a DMSO and EtOAc mixture or a mixture comprising DMSO.

In an embodiment, the mixture is a ratio of DMSO: EtOAc from about 1:1 to 1:9 or wherein the DMSO is at least 10%, at least 20%, at least 30%, at least 40% or at least or up to 50% DMSO.

In an embodiment, the ratio is about 1:9 DMSO: EtOAc or the DMSO is at least 10%.

In an embodiment, the acylation wash solvent is or comprises DMSO.

In an embodiment, the acylation wash solvent is a DMSO and EtOAc mixture or a mixture comprising DMSO.

In an embodiment, the mixture is a ratio of DMSO: EtOAc wherein the DMSO is greater than 30% or 35%, or is or is at least 35% or 40%, optionally wherein the ratio is from about 4:6 to 1:1.

In an embodiment, the ratio is about 1:1 DMSO: EtOAc or wherein the DMSO is at least 50%.

In an embodiment, the displacement solvent is or comprises DMSO or EtOAc.

In an embodiment, the displacement solvent is a DMSO and EtOAc mixture or a mixture comprising DMSO or a mixture comprising EtOAc.

In an embodiment, the mixture is a ratio of DMSO: EtOAc from about 10:1 to 1:9 or is a mixture comprising at least or about 10% DMSO.

In an embodiment, the ratio is about 1:9 DMSO: EtOAc or wherein the DMSO is at least or about 10%.

In an embodiment, the displacement wash solvent is or comprises DMSO or EtOAc.

In an embodiment, the displacement wash solvent is a DMSO and EtOAc mixture or a mixture comprising DMSO or a mixture comprising EtOAc.

In an embodiment, the displacement wash solvent comprises DMSO that is greater than 10%, 20%, 30% or 35%, or is at least 10%, 25%, 35% or 40%, optionally wherein the displacement wash solvent is a ratio of DMSO: EtOAc from about 1:9 to 1:1.

In an embodiment, the acylation wash solvent and the displacement wash solvent are the same solvent.

In an embodiment, the acylation solvent is EtOAc, the acylation wash solvent comprises a mixture of DMSO and EtOAc, the displacement solvent is or comprises EtOAc, and the displacement wash solvent is or comprises EtOAc.

In an embodiment, the acylation solvent is a mixture of DMSO and EtOAc, the acylation wash solvent is DMSO, the displacement solvent is a mixture of DMSO and EtOAc, and the displacement wash solvent is DMSO.

In an embodiment, the acylation solvent is about 1:9 DMSO: EtOAc, the acylation solvent wash is DMSO, the displacement solvent is about 1:9 DMSO: EtOAc, and the displacement wash solvent is DMSO.

In an embodiment, the acylation solvent is EtOAc, the acylation wash solvent is DMSO, the displacement solvent is EtOAc, and the displacement wash solvent is DMSO.

In an embodiment, the acylation solvent is EtOAc, the acylation solvent wash is about 1:1 DMSO: EtOAc, the displacement solvent is or comprises EtOAc, and the displacement wash solvent is about 1:1 DMSO: EtOAc.

In an embodiment, the acylation solvent is EtOAc, the acylation wash solvent is about 1:1 DMSO: EtOAc, the displacement solvent is EtOAc, and the displacement wash solvent is EtOAc.

In an embodiment, the acylation solvent is about 1:9 DMSO: EtOAc, the acylation wash solvent is about 1:1 DMSO: EtOAc, the displacement solvent is about 1:9 DMSO: EtOAc, and the displacement wash solvent is about 1:9 DMSO: EtOAc.

In an embodiment, the peptoid comprises a plurality of monomers of similar structure.

In an embodiment, the peptoid comprises a plurality of monomers of different structures.

In an embodiment, the deprotection wash solvent, acylation wash solvent, and displacement wash solvent are or comprise DMSO.

In an embodiment, the peptoid is synthesized manually.

In an embodiment, the peptoid is synthesized using an automated peptoid synthesizer.

In an embodiment, the peptoid is separated from the solid support, optionally a solid support resin, using a cleaving agent to obtain a crude peptoid.

In an embodiment, the solid support is a resin and is selected from a Rink Amide 4-methylbenzhydrylamine (MBHA) resin, a Rink Amide resin, a Wang resin, or a 2-chlorotrityl chloride resin.

In an embodiment, the solid support resin is the Rink Amide 4-methylbenzhydrylamine (MBHA) resin.

In an embodiment, prior to reacting a solid support, optionally a solid support resin comprising a terminal amine group, a protected terminal amine group is deprotected with a deprotection agent, optionally 4-methylpiperidine, in a deprotection solvent and washed with a deprotection wash solvent, to expose the terminal amine group.

In an embodiment, prior to deprotecting the terminal amine group, the solid support resin is solvated with a swelling solvent.

In an embodiment, the deprotection solvent is or comprises EtOAc or is or comprises DMSO.

In an embodiment, the deprotection solvent is a mixture of DMSO and EtOAc or a mixture comprising DMSO or a mixture comprising EtOAc.

In an embodiment, the ratio of DMSO: EtOAc is about 1:9 or the DMSO is or wherein the DMSO is at least 10%, at least 20%, at least 30%, at least 40% or at least or up to 50% DMSO.

In an embodiment, the deprotection wash solvent is or comprises DMSO or EtOAc or a mixture thereof. In an embodiment, the deprotection wash solvent is a mixture of DMSO and EtOAc.

In an embodiment, the mixture of DMSO and EtOAc is a ratio of DMSO: EtOAc from about 9:1 to 1:9.

In an embodiment, the deprotection solvent is about 1:9 DMSO: EtOAc, and the deprotection wash solvent is DMSO.

In an embodiment, the deprotection solvent is EtOAc, and the deprotection wash solvent is DMSO.

In an embodiment, the deprotection solvent is EtOAc, and the deprotection wash solvent is about 1:1 DMSO: EtOAc.

In an embodiment, the deprotection solvent is about 1:9 DMSO: EtOAc and the deprotection wash solvent is about 1:9 DMSO: EtOAc.

In another aspect, the invention includes a kit comprising:
(a) an acylation solvent,
(b) an acylation wash solvent,
(c) a displacement solvent, and/or
(d) a displacement wash solvent,
   wherein the acylation solvent is or comprises EtOAc, the acylation wash solvent is or comprises DMSO, the displacement solvent is or comprises DMSO or EtOAc, and the displacement wash solvent is or comprises DMSO or EtOAc.

The kit may further comprise instructions, for example directions on how to use the kit. The kit may further comprise one or more vials or other receptacles for containing the components of the kit. The acylation solvent, the acylation wash solvent, the displacement solvent and the displacement wash solvent can be any acylation solvent, acylation wash solvent, displacement solvent or displacement wash solvent described herein.

The preceding section is provided by way of example only and is not intended to be limiting on the scope of the present disclosure and appended claims. Additional objects and advantages associated with the methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

FIG. 1: Overview of solid-phase peptoid synthesis (SPS). A) General scheme of solid-phase peptoid synthesis with standard solvents highlighted in red. B) Solvent consumption for the synthesis of 6-mer and 18-mer peptoids synthesized. C) Hazardous solvents traditionally used in solid-phase peptoid synthesis and the proposed green solvents being investigated.

FIG. 2: Optical microscopy images and distribution plots of Rink Amide MBHA resin (0.62 mmol/g loading) dry and swollen with traditional and proposed green solid-phase peptoid synthesis solvents.

FIG. 3: Characterization of model 6-mer peptoids (Npes). A) Purity analysis (%) of crude acetylated 6-mer peptoids produced via manual solid-phase synthesis with traditional and green conditions. Purities were determined by the area under the curve using UHPLC analysis at 214 nm. UHPLC traces are offset for Methods 1-6 for clarity. B) MALDI-TOF mass spectrum of crude acetylated 6-mer peptoid synthesized with traditional DMF/NMP method. C) MALDI-TOF mass spectrum of crude acetylated 6-mer peptoid synthesized with greenest method, Method 6.

FIG. 4: UHPLC chromatograms and purities (%) of crude acetylated (A) 6-mer (Npe6) and (B) 18-mer ((Nme-Npe)₉) peptoids produced via automated solid-phase synthesis with a modified Method 6 (all 1:1 DMSO: EtOAc washes) and Method 6. Purities were determined by the area under the curve using UHPLC analysis at 214 nm. UHPLC traces are offset for clarity.

FIG. 5: UHPLC chromatogram at 214 nm of the crude acetylated 6-mer peptoid, Npe6, synthesized using the standard DMF/NMP solid-phase submonomer synthetic method.

FIG. 6: UHPLC chromatogram at 254 nm of the crude acetylated 6-mer peptoid, Npe6, synthesized using the standard DMF/NMP solid-phase submonomer synthetic method.

FIG. 7: MALDI-TOF mass spectrum of the crude acetylated 6-mer peptoid, Npe6, synthesized using the standard DMF/NMP solid-phase submonomer synthetic method.

FIG. 8: UHPLC chromatogram at 214 nm of the crude acetylated 6-mer peptoid, Npe6, synthesized using Method 1 (GVL for all steps of the solid-phase submonomer synthetic method).

FIG. 9: UHPLC chromatogram at 254 nm of the crude acetylated 6-mer peptoid, Npe6, synthesized using Method 1 (GVL for all steps of the solid-phase submonomer synthetic method).

FIG. 10: MALDI-TOF mass spectrum of the crude acetylated 6-mer peptoid, Npe6, synthesized using Method 1 (GVL for all steps of the solid-phase submonomer synthetic method).

FIG. 11: UHPLC chromatogram at 214 nm of the crude acetylated 6-mer peptoid, Npe6, synthesized using Method 2 (DMSO for all steps of the solid-phase submonomer synthetic method).

FIG. 12: UHPLC chromatogram at 254 nm of the crude acetylated 6-mer peptoid, Npe6, synthesized using Method 2 (DMSO for all steps of the solid-phase submonomer synthetic method).

FIG. 13: MALDI-TOF mass spectrum of the crude acetylated 6-mer peptoid, Npe6, synthesized using Method 2 (DMSO for all steps of the solid-phase submonomer synthetic method).

FIG. 14: UHPLC chromatogram at 214 nm of the crude acetylated 6-mer peptoid, Npe6, synthesized using Method 3 (DMSO for all steps of the solid-phase submonomer synthetic method except the acylation solvent, which was DMF).

FIG. 15: UHPLC chromatogram at 254 nm of the crude acetylated 6-mer peptoid, Npe6, synthesized using Method 3 (DMSO for all steps of the solid-phase submonomer synthetic method except the acylation solvent, which was DMF).

FIG. 16: MALDI-TOF mass spectrum of the crude acetylated 6-mer peptoid, Npe6, synthesized using Method 3 (DMSO for all steps of the solid-phase submonomer synthetic method except the acylation solvent, which was DMF).

FIG. 17: UHPLC chromatogram at 214 nm of the crude acetylated 6-mer peptoid, Npe6, synthesized using Method 4 (1:9 DMSO: EtOAc for all steps of the solid-phase submonomer synthetic method except the washes, which were DMSO).

FIG. 18: UHPLC chromatogram at 254 nm of the crude acetylated 6-mer peptoid, Npe6, synthesized using Method 4 (1:9 DMSO: EtOAc for all steps of the solid-phase submonomer synthetic method except the washes, which were DMSO).

FIG. 19: MALDI-TOF mass spectrum of the crude acetylated 6-mer peptoid, Npe6, synthesized using Method 4 (1:9 DMSO: EtOAc for all steps of the solid-phase submonomer synthetic method except the washes, which were DMSO).

FIG. 20: UHPLC chromatogram at 214 nm of the crude acetylated 6-mer peptoid, Npe6, synthesized using Method 5 (EtOAc for all steps of the solid-phase submonomer synthetic method except the washes, which were DMSO).

FIG. 21: UHPLC chromatogram at 254 nm of the crude acetylated 6-mer peptoid, Npe6, synthesized using Method 5 (EtOAc for all steps of the solid-phase submonomer synthetic method except the washes, which were DMSO).

FIG. 22: MALDI-TOF mass spectrum of the crude acetylated 6-mer peptoid, Npe6, synthesized using Method 5 (EtOAc for all steps of the solid-phase submonomer synthetic method except the washes, which were DMSO).

FIG. 23: UHPLC chromatogram at 214 nm of the crude acetylated 6-mer peptoid, Npe6, synthesized using Method 6 (EtOAc for all steps of the solid-phase submonomer synthetic method except the acylation wash, which was 1:1 DMSO: EtOAc).

FIG. 24: UHPLC chromatogram at 254 nm of the crude acetylated 6-mer peptoid, Npe6, synthesized using Method 6 (EtOAc for all steps of the solid-phase submonomer synthetic method except the acylation wash, which was 1:1 DMSO: EtOAc).

FIG. 25: MALDI-TOF mass spectrum of the crude acetylated 6-mer peptoid, Npe6, synthesized using Method 6 (EtOAc for all steps of the solid-phase submonomer synthetic method except the acylation wash, which was 1:1 DMSO: EtOAc).

FIG. 26: ¹H-NMR (400 MHZ, DMSO-d6) of bromoacetic acid at time=0, 3, and 7 days.

FIG. 27: ¹³C-NMR (400 MHZ, DMSO-d6) of bromoacetic acid at time=0, 3, and 7 days.

FIG. 28: ¹H-NMR (400 MHZ, CD₃OD) of insoluble 1,3-diisopropylurea by-product formed by reaction of BrAc and DIC in EtOAc. 0 (ppm) 4.37 (s, 2H), 3.77 (hept, J=6.5 Hz, 2H), 1.10 (d, J=6.6 Hz, 12H).

FIG. 29: ¹³C-NMR (400 MHZ, CD₃OD) of insoluble 1,3-diisopropylurea by-product formed by reaction of BrAc and DIC in EtOAc.

FIG. 30: UHPLC chromatogram at 214 nm of the crude acetylated 18-mer peptoid, Npe6, synthesized using EtOAc for all steps of the automated solid-phase submonomer synthetic method except the washes, which were 1:1 DMSO: EtOAc.

FIG. 31: UHPLC chromatogram at 214 nm of the crude acetylated 18-mer peptoid, Npe6, synthesized using EtOAc for all steps of the automated solid-phase submonomer synthetic method except the acylation wash, which was 1:1 DMSO: EtOAc.

FIG. 32: Solubility comparison of 10 mg/mL solutions of 1,3-diisopropylurea in various concentrations of DMSO and EtOAc.

FIG. 33: UHPLC characterization of 6-mer peptoids with different side chains (a-f). Purity analysis (%) of crude acetylated 6-mer peptoids produced via manual solid-phase synthesis with 1:9 DMSO: EtOAc for all steps, except the acylation wash (1:1 DMSO: EtOAc). Purities were determined by the area under the curve using UHPLC analysis at 214 nm. UHPLC traces are offset for (a-f) for clarity.

FIG. 34: UHPLC chromatogram at 214 nm of the crude acetylated 6-mer peptoid, Npe5Nce, synthesized using 1:9 DMSO: EtoAc for all steps of the manual solid-phase submonomer synthetic method except the acylation wash, which was 1:1 DMSO: EtOAc.

FIG. 35: MALDI-TOF chromatogram of the crude acetylated 6-mer peptoid, Npe5Nce, synthesized using 1:9 DMSO: EtoAc for all steps of the manual solid-phase submonomer synthetic method except the acylation wash, which was 1:1 DMSO: EtOAc.

FIG. 36: UHPLC chromatogram at 214 nm of the crude acetylated 6-mer peptoid, Npe5Nte, synthesized using 1:9 DMSO: EtoAc for all steps of the manual solid-phase submonomer synthetic method except the acylation wash, which was 1:1 DMSO: EtOAc.

FIG. 37: MALDI-TOF chromatogram of the crude acetylated 6-mer peptoid, Npe5Nte, synthesized using 1:9 DMSO: EtoAc for all steps of the manual solid-phase submonomer synthetic method except the acylation wash, which was 1:1 DMSO: EtOAc.

FIG. 38: UHPLC chromatogram at 214 nm of the crude acetylated 6-mer peptoid, Npe5Nall, synthesized using 1:9 DMSO: EtoAc for all steps of the manual solid-phase submonomer synthetic method except the acylation wash, which was 1:1 DMSO: EtOAc.

FIG. 39: MALDI-TOF chromatogram of the crude acetylated 6-mer peptoid, Npe5Nall, synthesized using 1:9 DMSO: EtoAc for all steps of the manual solid-phase submonomer synthetic method except the acylation wash, which was 1:1 DMSO: EtOAc.

FIG. 40: UHPLC chromatogram at 214 nm of the crude acetylated 6-mer peptoid, Npe5Nffe, synthesized using 1:9 DMSO: EtoAc for all steps of the manual solid-phase submonomer synthetic method except the acylation wash, which was 1:1 DMSO: EtOAc.

FIG. 41: MALDI-TOF chromatogram of the crude acetylated 6-mer peptoid, Npe5Nffe, synthesized using 1:9 DMSO: EtoAc for all steps of the manual solid-phase submonomer synthetic method except the acylation wash, which was 1:1 DMSO: EtOAc.

FIG. 42: UHPLC chromatogram at 214 nm of the crude acetylated 6-mer peptoid, Npe5Nhex, synthesized using 1:9 DMSO: EtoAc for all steps of the manual solid-phase submonomer synthetic method except the acylation wash, which was 1:1 DMSO: EtOAc.

FIG. 43: MALDI-TOF chromatogram of the crude acetylated 6-mer peptoid, Npe5Nhex, synthesized using 1:9 DMSO: EtoAc for all steps of the manual solid-phase submonomer synthetic method except the acylation wash, which was 1:1 DMSO: EtOAc.

### DETAILED DESCRIPTION OF VARIOUS EMBODIMENTS

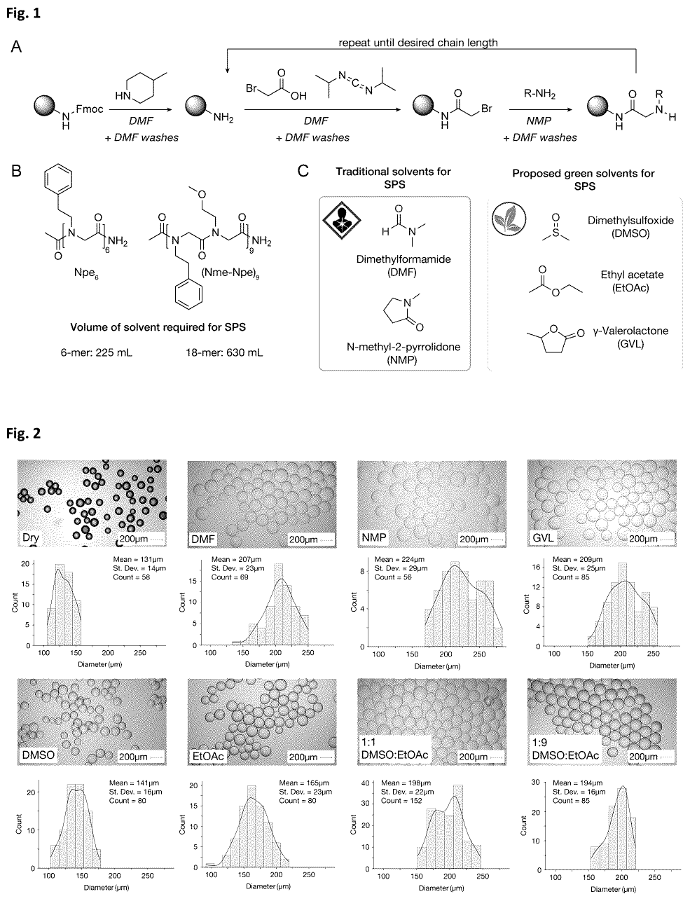

Described herein are methods and reagents that use greener solvents in the synthesis of peptoids (see for example FIG. 1C) . . . . As demonstrated herein, a variety of solvents were tested for their resin swelling performance using microscopy-based techniques and used as replacements for traditional solvents such as DMF and NMP in specific steps of the manual solid-phase synthesis of 6-mer polypeptoids. These peptoids were characterized by ultra-high-performance liquid chromatography (UHPLC) and matrix-assisted laser desorption/ionization time of flight mass spectrometry (MALDI-TOF MS) and crude purities were compared between the traditionally used solvents and greener solvents. Solid-phase synthesis of peptoids with complete elimination of DMF and NMP are disclosed herein, which attain for example the purities and yields attainable with traditional solvents. These methods offer a solution to produce peptoids in research and industrial applications with improved safety and sustainability.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

The term “about” as used herein may be used to take into account experimental error and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise. For example, a peptoid includes a plurality of peptoid oligomers.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”. For ranges described herein, subranges are also contemplated, for example every, 0.1 increment there between. For example, if the range is 80% to about 90%, also contemplated are 80.1% to about 90%, 80% to about 89.9%, 80.1% to about 89.9% and the like.

The term “N-substituted glycine polymers”, “peptoids” and peptoid “oligomers” (which are used interchangeably) refers to a class of poly (N-substituted amides), preferably a poly (N-Substituted glycines), as described, for example, in PCT Publications WO 94/06451, WO 98/06437, WO 99/08711, U.S. Pat. Nos. 5,877,278, and 9,073,977 each of which are herein incorporated by reference. Peptoids can be synthesized using the submonomer method pioneered by Zuckermann and colleagues., where peptoid monomers are built by sequential haloacetylation of an amine functionality attached to a solid support and subsequent displacement of the halogen using a primary amine. Methods for preparation are described in for example PCT Publications WO 94/06451, WO 98/06437, WO 99/08711, U.S. Pat. Nos. 5,877,278 and 9,073,977 as well as well as Zuckermann, R. N.; Kerr, J. M.; Kent, S. B. H.; Moos, W. H., Efficient Method for the Preparation of Peptoids Oligo (N-Substituted Glycines) by Submonomer Solid-Phase Synthesis. Journal of the American Chemical Society 1992, 114 (26), 10646-10647 (Zuckerman et al, 1992) (all of which are herein incorporated by reference).

The term “swelling ratio” as used herein, refers to the ratio of the average diameter of the swollen resin to the average diameter of the dry resin.

The term “solid support” as used herein refers to any amine or hydroxyl derivatized solid support, optionally amine-derivatized and protected, including a plurality of beads, planar surface or resin, such as a solid-phase resin with an Fmoc protecting group.

The term “solid support resin” as used herein refers to any amine or hydroxyl derivatized solid support resin, optionally amine-derivatized and protected, such as a solid-phase resin with an Fmoc protecting group. Examples include Rink Amide 4-methylbenzhydrylamine (MBHA) resin, a Rink Amide resin, a Wang resin, or a 2-chlorotrityl chloride resin.

### Methods and Kits

As aspect of the disclosure includes a method of synthesizing a peptoid, the method comprising
a) reacting a solid support, optionally a solid support resin, comprising a terminal amine group, with a haloacetic acid (HA) in the presence of a HA activating agent in an acylation solvent, to obtain a solid support resin comprising an acylated amino group;
b) washing the solid support, optionally a solid support resin, comprising the acylated amino group with an acylation wash solvent to obtain a washed solid support resin comprising the acylated amino group;
c) reacting the washed solid support, optionally a solid support resin, comprising the acylated amino group with a primary amine in a displacement solvent, to obtain a washed solid support comprising an amide acylated resin-bound amide;
d) washing the solid support, optionally a solid support resin, comprising the acylated resin-bound amide, with a displacement wash solvent, to obtain an attached peptoid monomer comprising an amine group;
e) repeating steps a)-d) at least once, where the amine group is the terminal amine group in step a) to obtain a peptoid;
wherein the acylation solvent, and the displacement solvent are polar aprotic solvents that are non-reactive to secondary amines and optionally the acylation solvent provides for a resin swelling of at least 1.25 and/or the acylation wash solvent is able to solubilize 1,3-diisopropylurea; or
wherein the acylation solvent is selected from EtOAc, or a DMSO and EtOAc mixture, or
wherein the acylation wash solvent is selected from DMSO, EtOAc, or combinations thereof, and wherein when the acylation solvent is EtOAc, the acylation wash solvent is not EtOAc, and when the acylation wash solvent is DMSO, the acylation solvent is not DMSO.

The terminal amine group and the acylated amino group can refer to the first resin bound groups or ones generated during repeating steps a) to d). For example, the first acylated resin-bound amide can be directly attached to the resin. As steps a)-d) are repeated and additional monomers are generated, the first acylated resin-bound amide can be in the growing chain and indirectly attached to the resin via the added monomers.

The acylation solvent, and displacement solvent are non-reactive to secondary amines meaning they are polar aprotic solvents (e.g., they do not participate in hydrogen bonding) that do not react covalently and/or for example non-reversibly with secondary amines. The acylation wash and the displacement wash solvents can also be polar aprotic solvents. In an embodiment, the acylation wash and the displacement wash solvents are also non-secondary amine reactive solvents. These various solvents can be for example DMSO, EtOAc and mixtures thereof or other solvents, including traditional solvents, for example, reduced amounts thereof, and mixtures of any of the foregoing.

As demonstrated herein, where the solid support is a resin, swelling of the resin is required for efficient synthesis. In an embodiment, the acylation solvent provides a resin swelling at a ratio of at least or about 1.25, 1.30, 1.35, 1.40, 1.45, 1.50, 1.55, up to a swelling ratio of about 1.60 or greater.

In various embodiments, the wash solvents including the acylation wash solvent and the displacement wash solvents or other wash solvents described herein may be a solvent that swells the resin and has a swelling ratio of at least 1.25, 1.30, 1.35, 1.40, 1.45, 1.50, 1.55, up to a swelling ratio of 1.60 or greater.

In some embodiments, the haloacetic acid is selected from a bromoacetic acid, chloroacetic acid, or iodoacetic acid. 13.43 In some embodiments, the haloacetic acid is bromoacetic acid.

The HA activating agent can for example be or comprise N, N′-diisopropylcarbodiimide (DIC).

The solid support can for example be a resin, typically comprising a Fmoc protected amide. For example, the resin can be selected from a Rink Amide 4-methylbenzhydrylamine (MBHA) resin, a Rink Amide resin, a Wang resin, or a 2-chlorotrityl chloride resin. Other resins can also be used.

Where the solid support comprises a hydroxyl derivatization, the hydroxyl can be converted to a Fmoc protected amide and the Fmoc protected amide deprotected, for example as described herein.

In some embodiments, the acylation solvent is or comprises EtOAc and/or DMSO. In some embodiments, the acylation solvent is a DMSO and EtOAc mixture. For example, the mixture can comprise as little as 1% DMSO, at least or up to 10% DMSO, up to 20% or any amount up to for example 30%, 35%, 40%, 45% or 50% DMSO. In some embodiments, the mixture comprises about 10% DMSO and 90% EtOAc (e.g., a ratio of 1:9 DMSO: EtOAc), to about 50% DMSO and 50% EtOAc (e.g., a ratio of 1:1 DMSO: EtOAc). The mixture of DMSO: EtOAc may comprise for example 40% DMSO and 60% EtOAc (e.g., a ratio of 4:6 DMSO: EtOAc), 30% DMSO and 70% EtOAc (e.g., a ratio of about 1:2 DMSO: EtOAc), 25% DMSO and 75% EtOAC (e.g. a ratio of 1:3 DMSO: EtOAc), 20% DMSO and 80% EtOAc (e.g. a ratio of 1:4 DMSO: EtOAc). In some embodiments, the mixture comprises about 10% DMSO and 90% EtOAc (e.g., a ratio of about 1:9 DMSO: EtOAc). In an embodiment, the acylation solvent is preferably not DMSO (e.g. not 100% DMSO) or not greater than 50%, 40%, 30% or 20% DMSO.

In some embodiments, the acylation wash solvent is or comprises DMSO or EtOAc. In some embodiments, the acylation wash solvent comprises DMSO. In some embodiments, the acylation wash solvent comprises greater than 30% DMSO. For example, it can comprise, at least 35% or at least 40% DMSO. In some embodiments, the acylation wash solvent is a DMSO and EtOAc mixture. For example, the DMSO EtOAc mixture, comprises greater than 30% DMSO or at least 35%, or at least 40% DMSO with the balance being EtOAc. In some embodiments, the mixture comprises greater than 30% DMSO and less than 70% EtOAc. In some embodiments, acylation solvent wash solvent is a ratio of DMSO: EtOAc from about 4:6 to 1:1. The mixture of DMSO: EtOAc may be a ratio of about 2:3 DMSO: EtOAc, 4:5 DMSO: EtOAc, 6:7 DMSO: EtOAc, 7:8 DMSO: EtOAc, 8:9 DMSO: EtOAc, or 9:10 DMSO: EtOAc. In some embodiments, the mixture comprises about 50% DMSO and 50% EtOAC (e.g., a ratio is about 1:1 DMSO: EtOAc).

The acylation wash solvent, for example, is one that can solubilize 1,3-diisopropylurea. As demonstrated in the Examples, solubilizing 1,3-diisopropylurea can be accomplished by using a acylation wash solvent that comprises DMSO. Solubilization of this byproduct is demonstrated for example when there is a lack of byproduct residue after 3 washes using a cellulose filter with a pore size of about 11 um. A wash or each wash, for example, may solubilize greater than 90% of the by-product.

In some embodiments, the displacement solvent is or comprises DMSO and/or EtOAc. In some embodiments, the displacement solvent is a DMSO and/or EtOAc mixture. For example, the displacement solvent can comprise any amount of DMSO or EtOAc, for example at least or about 10% DMSO. As shown in the Examples, reactions with the amount of DMSO at 10% are efficient. The range can be for example 1%-99% or any subrange therein.

In some embodiments, the mixture comprises about 10% DMSO and 90% EtOAc (e.g. a ratio of about 1:9 DMSO: EtOAc), to about 90% DMSO and 10% EtOAc (e.g. a ratio of about 9:1 DMSO: EtOAc). The mixture may comprise about 20% DMSO and 80% EtOAc (e.g. a ratio of about 2:8 DMSO: EtOAc), about 30% DMSO and 70% EtOAc (e.g. a ratio of about 3:7 DMSO: EtOAc), about 40% DMSO and 60% EtOAc (e.g. a ratio of about 4:6 DMSO: EtOAc), about 50% DMSO and 50% EtOAc (e.g. a ratio of about 1:1 DMSO: EtOAc), about 60% DMSO and 40% EtOAc (e.g. a ratio of about 6:4 DMSO: EtOAc), about 70% DMSO and 30% EtOAc (e.g. a ratio of about 7:3 DMSO: EtOAc), about 80% DMSO and 20% EtOAc (e.g. a ratio of about 8:2 DMSO: EtOAc). In some embodiments, the mixture comprises about 10% DMSO and 90% EtOAc (e.g. a ratio of about 1:9 DMSO: EtOAc).

In some embodiments, the displacement wash solvent is or comprises DMSO or EtOAc. The displacement wash solvent can comprise any amount of DMSO e.g., 0-100% or any amount of EtOAc, e.g., 0-100% or any subrange therein. Other wash solvents can also be used.

In some embodiments, the displacement wash solvent is a DMSO and EtOAc mixture.

In some embodiments, the acylation wash solvent and the displacement wash solvent are the same.

In some embodiments, the acylation solvent is EtOAc, the acylation wash solvent comprises a mixture of DMSO and EtOAc, the displacement solvent is EtOAc, and the displacement wash solvent is or comprises EtOAc.

In some embodiments, the acylation solvent is a mixture of DMSO and EtOAc, the acylation solvent wash is DMSO, the displacement solvent is a mixture of DMSO and EtOAc, and the displacement wash solvent is DMSO.

In some embodiments, the displacement wash solvent is a mixture comprising 10% DMSO and 90% EtOAc (e.g., a ratio of 1:9 DMSO: EtOAc) to a mixture comprising about 90% DMSO and 10% EtOAc (e.g., a ratio of 9:1 DMSO: EtOAc). The mixture may further comprise a mixture of about 50% DMSO and 50% EtOAc (e.g., a ratio of 1:1 DMSO: EtOAc). The mixture may further comprise a mixture of about 60% DMSO and 40% EtOAc (e.g., a ratio of 6:4 DMSO: EtOAc), 70% DMSO and 30% EtOAc (e.g., a ratio of 7:3 DMSO: EtOAc), or 80% DMSO and 20% EtOAc (e.g., a ratio of 8:2 DMSO: EtOAc).

In some embodiments, the acylation solvent is about 1:9 DMSO: EtOAc, the acylation solvent wash is DMSO, the displacement solvent is about 1:9 DMSO: EtOAc, and the displacement wash solvent is DMSO.

In some embodiments, the acylation solvent is EtOAc, the acylation wash solvent is DMSO, the displacement solvent is EtOAc, and the displacement wash solvent is DMSO.

In some embodiments, the acylation solvent is EtOAc, the acylation solvent wash is about 1:1 DMSO: EtOAc, the displacement solvent is or comprises EtOAc, and the displacement wash solvent is about 1:1 DMSO: EtOAc.

In some embodiments, the acylation solvent is EtOAc, the acylation wash solvent is about 1:1 DMSO: EtOAc, the displacement solvent is EtOAc, and the displacement wash solvent is EtOAc.

In some embodiments, the acylation solvent is about 1:9 DMSO: EtOAc, the acylation wash solvent is about 1:1 DMSO: EtOAc, the displacement solvent is about 1:9 DMSO: EtOAc, and the displacement wash solvent is about 1:9 DMSO: EtOAc.

In general, the ratios of solvents and solvent percentages when referring to liquids described herein denote volume/volume (v/v).

Reference to a solvent being (or not being) a particular solvent e.g., wherein the solvent is DMSO, means for example that the solvent is (or is not) 100% or is (or is not) about 100%, for example at least 95%, 96%, 97%, 98%, 99% or more the named solvent. For example, it can comprise trace amounts of other solvents or impurities.

Various primary amines can be used as known in the art. For example, any commercially or synthetically available amine can be incorporated using the methods described herein. A plurality of the same primary amine can be used to obtain a peptoid with the same repeating monomer. Alternatively, for example a plurality of different primary amines can be used to obtain a peptoid with different, or alternating monomer units. As such, the peptoid is highly tunable, depending on the structure and function of the primary amines used. As shown in the Examples, 2-phenylethylamine and 2-methoxyethylamine were used to make peptoids comprising N-(2-methoxyethyl) glycine (Nme) and/or poly (N-2-phenethylglycine) (Npe) monomers. As further shown in Example 8, primary amines with protecting groups (e.g., a primary amine comprising a carboxylic acid), increased hydrophilicity (e.g., a primary amine comprising a repeating-[O—CH₂—CH₂—]-chain), capacity for post-functionalization (e.g. a primary amine comprising an alkenyl), heterocyclic functional groups (e.g. a primary amine comprising a furan ring), and amines with increased hydrophobicity (e.g., a primary amine comprising an alkyl chain), can be used to synthesize peptoids using the present methods. In an embodiment the primary amine comprises t-butyl beta-alanine (optionally beta-alanine tert-butyl ester (CAS NO: 58620-93-2)), 2-(2-(2-methoxyethoxy) ethoxy) ethanamine, allylamine, or furfurylamine.

Peptoids can be synthesized according to the presently described methods manually or on an automated synthesizer for example using a modified version of the Solid-phase Submonomer method described in Zuckermann, 1992 or Example 5. For example, the Fmoc group on a Rink amide resin can be deprotected with piperidine in a solvent such as DMF or using a solvent described herein. Peptoid synthesis can involve a solution of bromoacetic acid in a solvent described herein such as EtOAc or mixtures of EtOAc and DMSO and N, N′-diisopropylcarbodiimide (DIC) added to a resin-bound amine and mixed for example for about 20 min or longer at for example 35° C. (e.g. acylation step of the submonomer cycle). The resin-bound bromide can then be displaced with the amine submonomer by adding a solution of the amine in a solvent described herein. This displacement reaction can be carried out a suitable condition, such as for 90 min or longer at for example 35° C. The crude peptoid products can be cleaved from the resin with a suitable solvent, such as 95:5 trifluoroacetic acid (TFA)/water (v/v), under suitable conditions, such as for 20 min at room temperature. The cleavage solution can be filtered and evaporated under a stream of inert gas, such as nitrogen, to remove the TFA. The crude peptoid product can then be dissolved in a suitable solvent, such as a mixture of water and acetonitrile, and subjected to further purification, such as reverse-phase HPLC. The peptoids can be lyophilized, dissolved in a suitable solvent, such as either water or buffer (40 mM sodium phosphate buffer, pH 7.0), and stored for example at −70° C.

Various automatic synthesizers can be used, for example the Gyros Protein Technologies PurePep® Chorus®. Exemplary methods that can be used with manual and automatic synthesizers are disclosed in the Examples. The methods may comprise one or more of the steps described in the Examples, for example in any of Examples 1, 3, 4 and/or 5. In some embodiments, the method uses a single type of wash solvent for each of the wash solvents (e.g., deprotection wash solvent, acetylation wash solvent, displacement wash solvent, etc.), as disclosed in Example 5 (Table 4).

As mentioned, the peptoids are tunable and the methods can be used to generate any kind of peptoid. In some embodiments, the peptoid comprises a plurality of monomers of similar structure. In some embodiments, the peptoid comprises a plurality of monomers of different structures.

The peptoid synthesized can be any length of monomers, for example 10, 20, 50, 100 monomers or longer.

One or more, or each of the washing steps can comprise one or more washes for example, 2, 3, 4 or 5 or more washes.

In some embodiments, the peptoid is separated from the solid support using a cleaving agent to obtain a crude peptoid. The cleaving agent may be for example trifluoroacetic acid (TFA). Additionally, the cleaving agent may be various cleaving cocktails, for example, such as those listed in references Palomo, J. M. Solid-Phase Peptide Synthesis: An Overview Focused on the Preparation of Biologically Relevant Peptides. RSC Adv. 2014, 4 (62), 32658-32672; King, D. S.; Fields, C. G.; Fields, G. B. A Cleavage Method Which Minimizes Side Reactions Following Fmoc Solid Phase Peptide Synthesis. Int. J. Pept Protein Res. 1990, 36 (3), 255-266; or Stawikowski, M.; Fields, G. B. Introduction to Peptide Synthesis. Curr. Protoc. Protein Sci. 2012, 69 (1), 18.1.1-18.1.13. The crude peptoid refers to the peptoid after cleavage and before further purification. The crude peptoid may have a purity of 50%, 55%, 60%, 65%, 70%, 75%, 79%, 80%, 85%, 90%, 92%, 95% or 99%. For example, as demonstrated in Example 4 (Method 4), a crude purity of 92% was obtainable using the methods described herein. In another example, as demonstrated in Example 4 (Methods 5 and 6), a crude purity of 79% was obtainable using the methods described herein.

The crude peptoid may be further modified, for example the crude peptoid may be acetylated. Additionally, the crude peptoid may be purified using, for example reverse-phase high performance liquid chromatography. The crude peptoid may also be purified using, for example recycling gel permeation chromatography in chloroform. Other purification modalities are contemplated.

In some embodiments, prior to reacting a solid support, optionally a solid support resin, comprising a terminal amine group, a protected terminal amine group is deprotected with a deprotection agent, optionally 4-methylpiperidine, in a deprotection solvent and washed with a deprotection wash solvent, to expose the terminal amine group.

In some embodiments, prior to deprotecting the terminal amine group, the solid support, optionally solid support resin, is solvated with a swelling solvent. The swelling agent can be any suitable agent that provides a swelling ratio of at least 1.25, 1.3, 1.35, 1.4, 1.45, 1.5 or 1.55 up to a swelling ratio of about 1.60 or greater.

For example, the deprotection solvent can be or comprise EtOAC or can be or comprises DMSO. Various ratios and percentages including those described for other washes can be used.

In some embodiments, the deprotection solvent is a mixture of DMSO and EtOAC. In some embodiments, the ratio of DMSO: EtOAc is about 1:9. The deprotection solvent may be, for example, any mixture of DMSO and EtOAc. In some embodiments, the deprotection wash solvent is or comprises DMSO, or EtOAc. In some embodiments, the deprotection wash solvent is a mixture of DMSO and EtOAc. In some embodiments, the mixture of DMSO and EtOAc is a ratio from about 9:1 to 1:9 DMSO: EtOAc.

In some embodiments, the deprotection solvent is about 1:9 DMSO: EtOAc, and the deprotection wash solvent is DMSO. In some embodiments, the deprotection solvent is EtOAc, and the deprotection wash solvent is DMSO. In some embodiments, the deprotection solvent is EtOAc, and the deprotection wash solvent is about 1:1 DMSO: EtOAc. In some embodiments, the deprotection solvent is about 1:9 DMSO: EtOAc, and the deprotection wash solvent is about 1:9 DMSO: EtOAc.

In an embodiment, the deprotection solvent, the deprotection wash solvent, acylation solvent, the displacement solvent and/or the displacement wash solvent can be any ratio of DMSO: EtOAc. In an embodiment, the acylation wash solvent is DMSO, or a mixture comprising at least 35% or 40% DMSO. In embodiment, the acylation solvent comprises at least 10% DMSO and is optionally 10% DMSO and 90% EtOAc.

In some embodiments, when the acylation solvent is EtOAc (e.g., 100% EtOAc), the acylation wash solvent is not EtOAc (e.g., not 100% EtOAc), but may be for example, DMSO (e.g., 100% DMSO) or a mixture of DMSO and EtOAc. For example, as shown in the Examples e.g., Examples 3, 4, 5 and 6, (e.g., Table 1-Method 6), high yield synthesis is attainable using an EtOAc as the acylation solvent and a ratio of about 1:1 DMSO: EtOAc for the acylation wash solvent.

In some embodiments, when the displacement solvent is DMSO, the acylation solvent is not DMSO. When the displacement solvent is DMSO, in some embodiments, the acylation solvent is not DMSO.

In some embodiments, when the displacement solvent is EtOAc, the acylation wash solvent is not EtOAc. When the displacement solvent is EtOAc, the acylation wash solvent is not EtOAc, but may be DMSO or a mixture of DMSO and EtOAc

In some embodiments, when the displacement wash solvent is DMSO, the acylation solvent is not DMSO. For example, when the displacement wash solvent is DMSO, the acylation solvent is not DMSO, but may be EtOAc or a mixture of DMSO and EtOAc.

In some embodiments, when the displacement wash solvent is EtOAc, the acylation wash solvent is not EtOAc. For example, when the displacement wash solvent is EtOAc, the acylation wash solvent is not EtOAc, but may be a mixture of DMSO and EtOAc.

Traditional solvents such as N, N′-dimethylformamide (DMF), dichloromethane (DCL) or N-methyl-2-pyrrolidone (NMP) can be used for one or more steps. Replacing one or more steps with a greener solvent allows for reduction of toxic solvent use. Other solvents that comprise the properties described herein can also be used for one or more of the solvents or wash solvents.

Also provided herein is a peptoid produced using a method described herein.

Also provided is a solvent or solvent mixture described herein for use in a method described herein.

In another aspect, the invention includes a kit comprising:
(a) an acylation solvent,
(b) an acylation wash solvent,
(c) a displacement solvent, and/or
(d) a displacement wash solvent,
   wherein the acylation solvent is or comprises EtOAc, the acylation wash solvent is or comprises DMSO, the displacement solvent is or comprises DMSO or EtOAc, and the displacement wash solvent is or comprises DMSO or EtOAc.

The kit may further comprise instructions, for example directions on how to use the kit. The kit may further comprise one or more vials or other receptacles for containing the components of the kit.

In some embodiments, the kit comprises 2, 3, or 4 of the solvents listed.

The acylation solvent, the acylation wash solvent, the displacement solvent and the displacement wash solvent can be any acylation solvent, acylation wash solvent, displacement solvent or displacement wash solvent described herein. For example, each solvent can be provided in a ratio described herein. Further the kit can comprise multiple of one or more or each of the acylation solvent, the acylation wash solvent, the displacement solvent and the displacement wash solvent allowing selection of solvent depending on the desired method.

The kit may also comprise additional solvents or other components such as one or more of: one or more primary amines (optionally one or more different primary amines), a haloacetic acid, an activation agent such as DIC, and/or a suitable solid support (e.g., a resin or other solid support comprising with terminal amines, optionally protected such as Fmoc protected). The kit can also comprise deprotection solvent or deprotection wash solvent. The kit can also include any other component, eg. compound, solvent or reagent useful for making peptoids, including for example other components described herein.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1: Methods

### Reagents

All reagents and solvents were purchased from Sigma-Aldrich or Fisher Scientific and used without further purification. Rink Amide 4-methylbenzhydrylamine (MBHA) resin (0.62 mmol/g loading, Cat #: RAM-25-HL) was purchased from Gyros Protein Technologies and used for all solid-phase syntheses.

### Peptoid Synthesis

Manual solid-phase submonomer peptoid synthesis was performed in 25 mL glass peptide synthesis vessels (medium frit, T-Bore PTFE stopcock) purchased from Chemglass Life Sciences (Item #: CG-1866-02). Automated peptoid synthesis was performed using a Gyros Protein Technologies PurePep® Chorus® in 10 mL frosted glass reaction vessels. Filtering of the resin following cleavage was accomplished with 10 mL disposable reaction vessels purchased from Gyros Protein Technologies.

### UHPLC Analysis

Ultra-high-performance liquid chromatography (UHPLC) analyses were performed on a PerkinElmer Altus™ A-30 UPLC system (equivalent to a Waters Acquity H-Class UHPLC instrument) with a C18 50×2.1 mm i.d., 1.7 μm column and an ultra-violet (UV) detector. Peptoid samples were analyzed using a flow rate of 0.5 mL/min, UV detection at 214 and 254 nm, injection volume of 2.5 μL, and a concentration of 1 mg/mL. Solvent A: 95:5 (v/v) water: acetonitrile, solvent B: acetonitrile. Method: Linear gradient of 0%-100% B in 12 minutes. Data acquisition and processing were conducted using Waters Empower 3 software. Peaks below 2 minutes were excluded from the purity calculations as they elute earlier than any peptoid fragment and likely correspond to salts, solvents, or changes in the pH.

### MALDI-TOF Mass Spectrometry

Matrix-assisted laser desorption/ionization time-of-flight (MALDI-TOF) analyses were performed on a Bruker AutoFlex Speed MALDI-TOF mass spectrometer. All samples were analyzed using the conventional dried droplet method with a ground steel target using a 1:1 (v/v) mixture of polypeptoid (2-5 mg/mL in 1:1 acetonitrile: water) and 2,5-dihydroxybenzoic acid matrix dissolved in 1:1 acetonitrile: water at 10 mg/mL. Scan mode: linear; ionization mode: positive. Red phosphorous was used as the calibrant.

### NMR Spectroscopy

Nuclear magnetic resonance (NMR) spectra were acquired on a 400 MHZ Bruker Avance III NMR Spectrometer (v (1H)=400.22 Hz). 1D ¹H spectra were acquired using a zg45 pulse sequence at 25° C., a 1.0000 s recycle delay, and 16 transients. 1D ¹³C spectra were acquired using a zgpg35 pulse sequence at 25° C., a 0.2000 s recycle delay, and 512 transients. NMR Processing was carried out using MestreNova software (v 14. 3.0-30573).

All ¹H-NMR spectra were Fourier transformed with 0.2222 Hz exponential line broadening (0.5000 Hz for ¹³C-NMR), phased, and then baseline corrected. Chemical shifts were referenced relative to residual solvent peaks (DMSO, δ(1H)=2.50 ppm, δ(13C)=39.52 ppm; CD₃OD, δ(1H)=3.31 ppm, δ (13C)=49.00 ppm).

### Recycling GPC

Purification of peptoids was performed using a JAI LaboACE LC-7080 recycling preparative gel permeation chromatography (GPC) system equipped with JAIGEL-2.5 HR and JAIGEL-3 HR columns (20 mm i.d., 600 mm length) and dual UV and RI detectors using ACS reagent grade chloroform (contains 0.5-1.0% ethanol as stabilizer).

### Light Microscopy

Optical microscopy images were acquired on a Zeiss AxioObserver.Z1 inverted stand microscope with an X-Cite 120Q metal halide fluorescence lamp and a Hamamatsu ORCA-Flash 4.0 (4 MP sCMOS) camera. Images were initially processed using Zeiss Zen software and then analyzed using ImageJ.

### Example 2: Resin Swelling Studies in Traditional and Green Peptoid Synthesis Solvents

Methods used are described in Example 1 and herein.

10 mg of Rink Amide 4-methylbenzhydrylamine (MBHA) (0.62 mmol/g loading) were swollen in 2 mL of solvent for 1 hour with occasional agitation prior to analysis by light microscopy. The swelling performance of the selected greener solvents and their mixtures (GVL, DMSO, and EtOAc) were tested alongside traditional solvents (DMF and NMP), to assess their suitability for solid-phase peptoid synthesis. The Rink amide-MBHA resin was imaged dry and swollen in the various solvents. The swollen resin beads and solvent were placed on a dry glass slide and ImageJ software was used to measure the diameters of the randomly selected resin beads from images taken using an optical microscope. An average diameter was calculated for each solvent condition, as depicted in FIG. 2. Swelling ratios were determined from the change in resin bead diameter from the dry to swollen state and are reported in Table 2. It should be noted that in order to mimic conditions representative of the standard practice of solid-phase peptoid synthesis, the resin beads were not passed through a size filter before conducting the swelling experiments. The standard deviations for each swelling experiment are described in FIG. 2. Statistical analyses and distribution plots were obtained using Python.

The results disclosed that poor swelling of the resin causes decreased access to reaction sites within the resin for the deprotection, acylation, and displacement steps, resulting in reduced yields.

The average diameters of the resin beads were highest when treated with the traditional solid-phase solvents, NMP and DMF, as well as the green solvent, GVL. An acceptable solvent for solid-phase peptoid synthesis should swell the resin to ±30% of the diameter observed in the DMF condition. 30.31 Based on this criterion, only DMSO fell outside the desired swelling performance, with an observed 8% increase in diameter from the dry state (58% for DMF) (FIG. 1 and Table 2). However, the mixtures of DMSO with EtOAc (1:1 and 1:9) were more suitable for the solvation of the resin, showing increased swelling ratios compared to those exhibited by the individual solvents (Table 2). Mixtures of solvents can have different physical properties than their individual counterparts, which we hypothesize translates to different swelling efficiencies for the Rink Amide MBHA resin.

Table 2: Calculated swelling ratios of Rink amide-MBHA resin swollen in various solvents after 1 hour incubation. The swelling ratio is defined as the ratio of the average diameter of the swollen resin to the average diameter of the dry resin.

| Solvent | Swelling ratio |
|---|---|
| DMF | 1.58 |
| NMP | 1.71 |
| DMSO | 1.08 |
| EtOAc | 1.26 |
| GVL | 1.60 |
| 1:1 DMSO:EtOAc | 1.51 |
| 1:9 DMSO:EtOAc | 1.48 |

Diameters were determined by optical microscopy, as seen in FIG. 2.

### Example 3: General Procedure for Manual Solid-Phase Submonomer Peptoid Synthesis

Resin swelling: 100 mg of Rink amide-MBHA resin (0.62 mmol/g loading) was added to a 25 mL solid-phase peptide synthesis fritted reaction vessel. 2 mL of the swelling solvent (Table 1) was added and agitated by bubbling for 10 minutes and then drained.

Fmoc deprotection: 1 mL of 20% 4-methylpiperidine solution (Table 1) was added, agitated by bubbling for 2 minutes, and then drained. Another 1 mL of the 20% 4-methylpiperidine solution (deprotection solvent in Table 1) was added, agitated by bubbling for 12 minutes, and then drained. The resin was washed with 3×2 mL of the deprotection wash solvent (Table 1), agitated by bubbling for 15 seconds, and then drained.

Submonomer cycle: The addition of each monomer consists of a bromoacetylation reaction and a subsequent displacement step with a primary amine. Since our target peptoid was Npe6, the submonomer cycle was repeated for a total of six times using phenethylamine as the primary amine.

1 mL of 0.8 M bromoacetic acid solution (acylation solvent in Table 1) was added, followed by 1 mL of 0.8M N, N′-diisopropylcarbodiimide (DIC) (acylation solvent in Table 2). The solution was agitated by bubbling for 30 minutes and then drained.

The resin was washed with 3×2 mL of the acylation wash solvent (Table 1), agitated by bubbling for 15 seconds, and then drained.

1.5 mL of 1 M phenethylamine (displacement solvent in Table 1) was added to the reaction vessel, agitated by bubbling for 30 minutes, and then drained.

The resin was washed with 3×2 mL of the displacement wash solvent (Table 1), agitated by bubbling for 15 seconds, and then drained.

End of synthesis: After the final displacement is complete, the resin was washed with 3×2 mL of dichloromethane (DCM) and dried for 30 minutes. The resin was stored in a fridge until ready for cleavage.

Pause in synthesis: To pause the synthesis after the displacement reaction was complete, the resin was rinsed with 3×2 mL of the displacement wash solvent (Table 1), agitated by bubbling for 15 seconds, and then drained.

Continue growing the chain: To continue growing the chain after a pause in synthesis, the dried resin was re-swelled in 2 mL of the swelling solvent (Table 1) for 10 minutes.

Cleavage: The resin was transferred to a 20 mL scintillation vial, 4 mL of TFA was added and the solution was shaken for 30 minutes at room temperature. The solution was then filtered through a 10 mL disposable solid-phase peptide synthesis cartridge into a vial. The resin was rinsed with TFA to collect residual product. The TFA was then removed by blowing a gentle stream of air in the fume hood. The final crude peptoids were weighed and stored in the freezer.

Acetylation: The crude peptoids were acetylated by adding 4 equivalents of triethylamine and 8 equivalents of acetic anhydride to the crude peptoid in ethyl acetate (0.2M). The solution was shaken overnight and the solvent was removed by rotary evaporation.

TABLE 1
Purities and yield of 6-mer peptoids produced via manual solid-
phase peptoid synthesis with traditional and green conditions.

| | DMF/NMP | Method 1 | Method 2 | Method 3 | Method 4 | Method 5 | Method 6 |
|---|---|---|---|---|---|---|---|
| Swelling | DMF | GVL | DMSO | DMSO | 1:9 D:E | EtOAc | EtOAc |
| Deprotection | DMF | GVL | DMSO | DMSO | 1:9 D:E | EtOAc | EtOAc |
| Deprotection | DMF | GVL | DMSO | DMSO | DMSO | DMSO | EtOAc |
| Wash | | | | | | | |
| Acylation | DMF | GVL | DMSO | DMF | 1:9 D:E | EtOAc | EtOAc |
| Acylation | DMF | GVL | DMSO | DMSO | DMSO | DMSO | 1:1 D:E |
| Wash | | | | | | | |
| Displacement | NMP | GVL | DMSO | DMSO | 1:9 D:E | EtOAc | EtOAc |
| Displacement | DMF | GVL | DMSO | DMSO | DMSO | DMSO | EtOAc |
| Wash | | | | | | | |
| Cleavage | TFA | | | | | | |
| Acetylation | EtOAc | | | | | | |
| Crude purity^{a} | 95% | 56% | 0% | 80% | 92% | 79% | 79% |
| Yield^{b} | 51% | —^{c} | —^{c} | —^{c} | —^{c} | —^{c} | 51% |

The abbreviations used in Table 1: 1:9 DMSO:EtOAc (1:9 D:E), 1:1 DMSO:EtOAc (1:1 D:E), trifluoroacetic acid (TFA).
^{a}Crude purity determined by UHPLC based on area % of peak at ~8.5 min.
^{b}Lower yields are due to the nature of the purification by recycling GPC.
^{c}Yields were only determined for the non-acetylated peptoids synthesized using the traditional and greenest conditions.

### Example 4: Green Solid-Phase Manual Synthesis of Model 6-Mer Peptoid

Methods used are described in Examples 1 and 3 and herein.

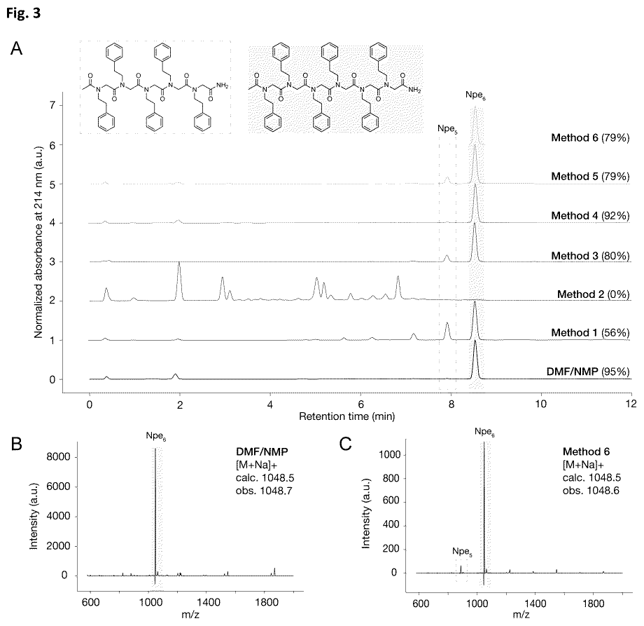

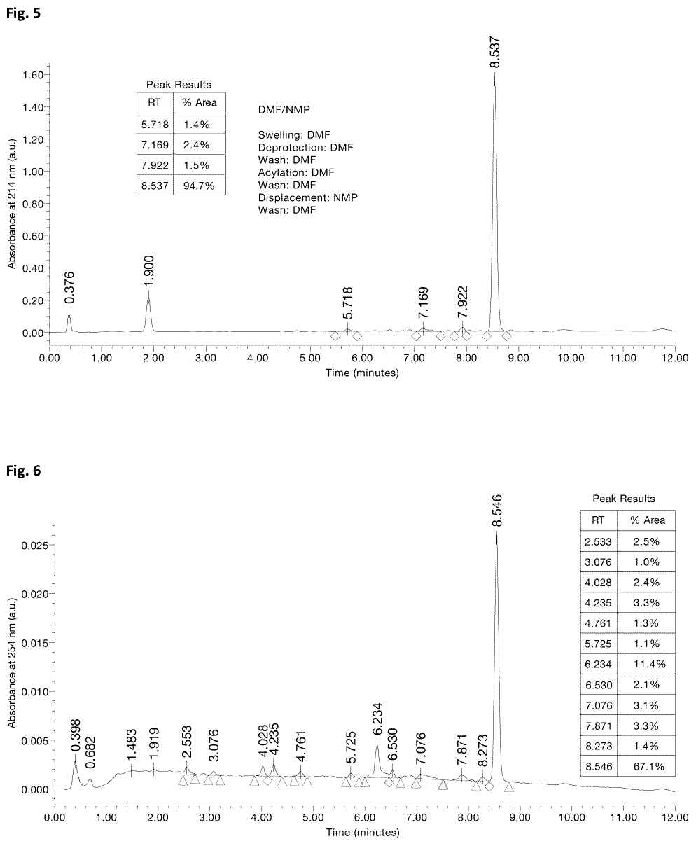

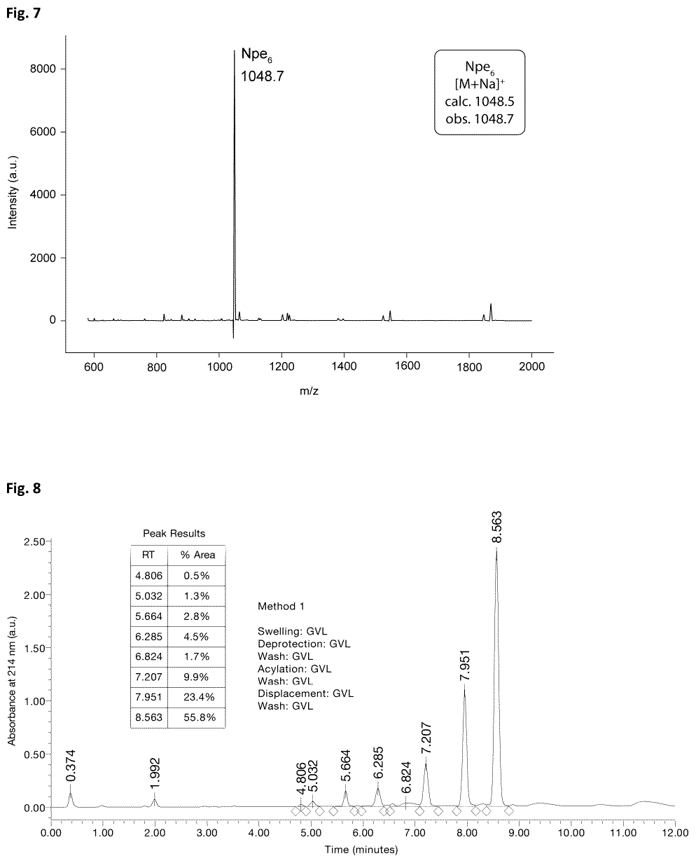

Once the selected solvents were screened for their resin swelling performance, their compatibility with solid-phase submonomer peptoid synthesis was investigated. These green solvents were used to synthesize a model 6-mer peptoid, poly (N-2-phenethylglycine) (Npe6) (Scheme S1).³²⁻³⁴ A traditional synthesis in DMF and NMP was conducted as a control for comparison of purity and yield relative to the green solvents (FIG. 3A-B, FIGS. 5-7). DMF and NMP were substituted with the new solvents at various steps of solid-phase peptoid synthesis, as described by the six methods in Table 1.

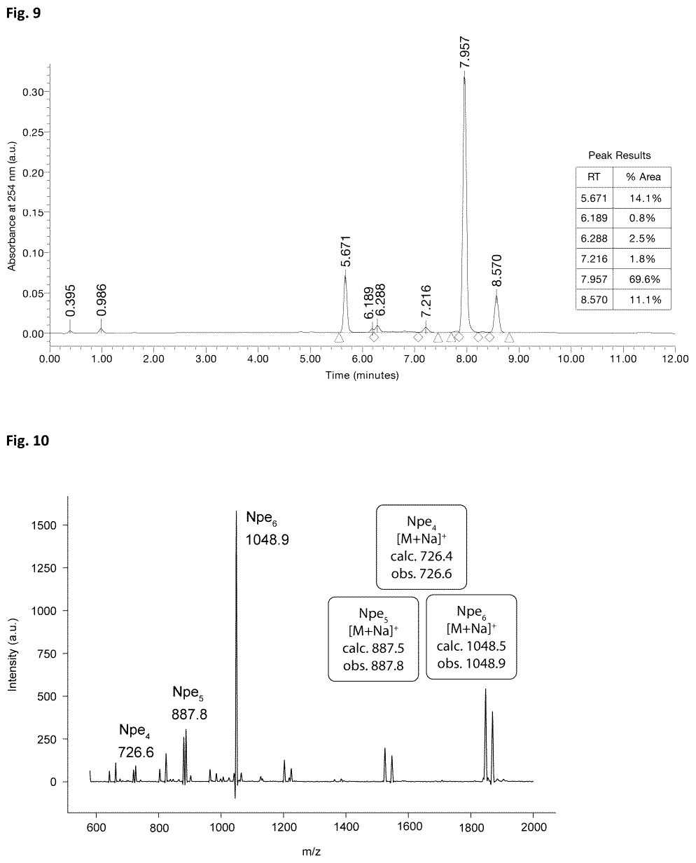

GVL, a biomass derived solvent successful in green peptide synthesis, was first investigated as an alternative to DMF and NMP for all steps of solid-phase synthesis (Method 1 in Table 1).35 The GVL method yielded the target 6-mer polypeptoid with lower overall purity compared to the traditional DMF/NMP protocol, as determined by % peak area by UHPLC (FIG. 3A). The peaks included in the purity calculation are highlighted in FIG. 8. Note that any peaks with retention times below 2 minutes were excluded from all UHPLC purity calculations as they elute earlier than any possible peptoid fragment. Several side products formed during the peptoid synthesis in GVL, resulting in the additional peaks observed in the UHPLC chromatogram (FIG. 3A and FIGS. 8-9). Mass analysis by MALDI-TOF revealed that the peak at 7.95 min corresponded to the 5-mer (observed=887.8, calculated=887.5) and the peak at 7.21 min corresponded to the 4-mer (observed=726.6, calculated=726.4) (FIG. 10). Without wishing to be bound to theory, it is hypothesized that in solid-phase peptoid synthesis, the GVL may undergo a ring-opening reaction with 4-methylpiperidine (4-me-pip) during the resin deprotection, with the primary amine solution, as well as with the growing end of the peptoid chain following displacement. This could lead to incomplete deprotection of the resin as well as early termination of the peptoid chains, contributing to the shorter chain polypeptoids observed for this method. The swelling experiments indicated that the inefficient conversion to the 6-mer is likely not due to poor swelling of the resin with GVL as its swelling ratio was comparable to DMF (FIG. 2, Table 2). While GVL has been successful for solid-phase peptide synthesis, it does not translate well for solid-phase peptoid synthesis.

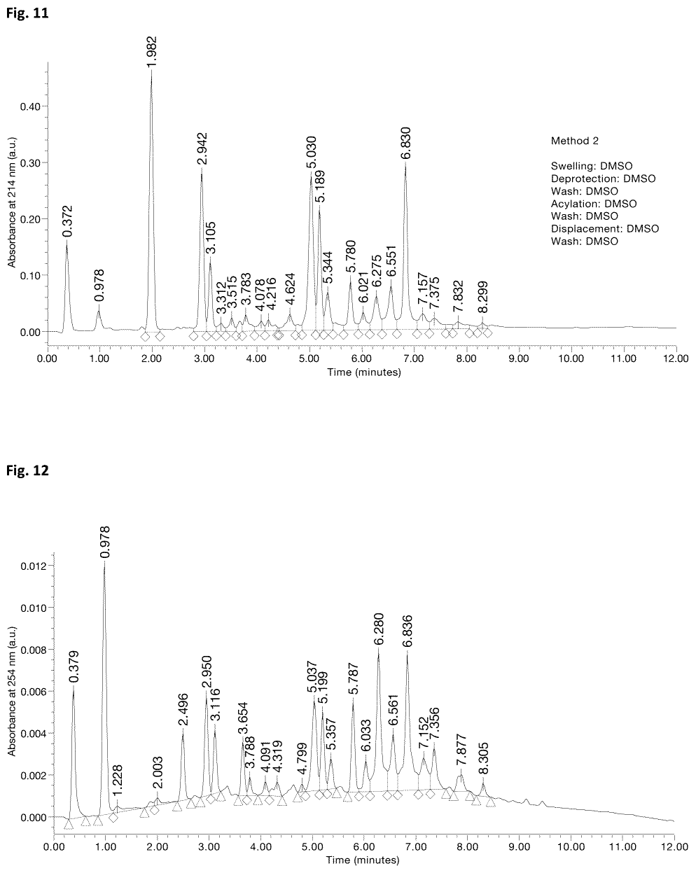

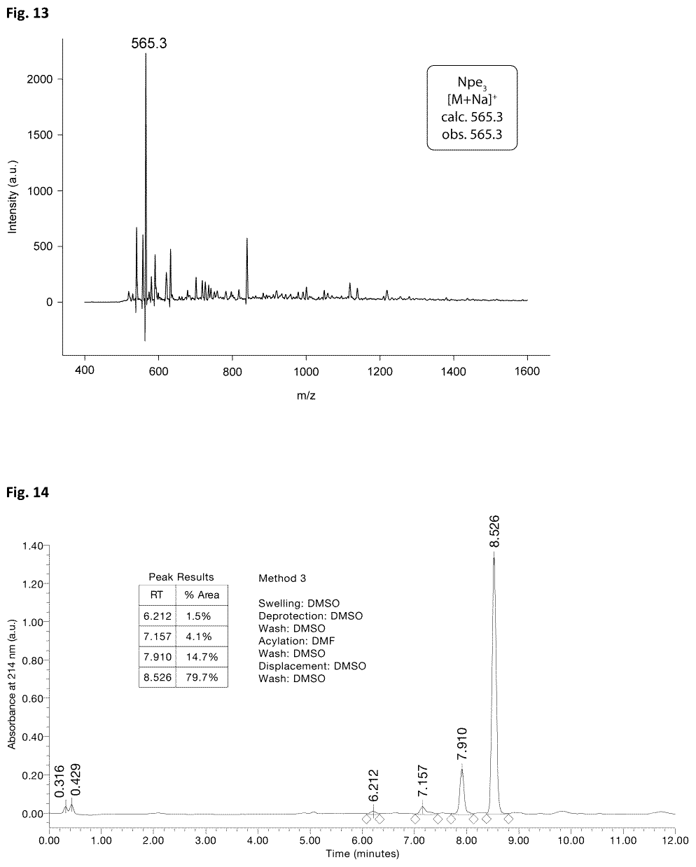

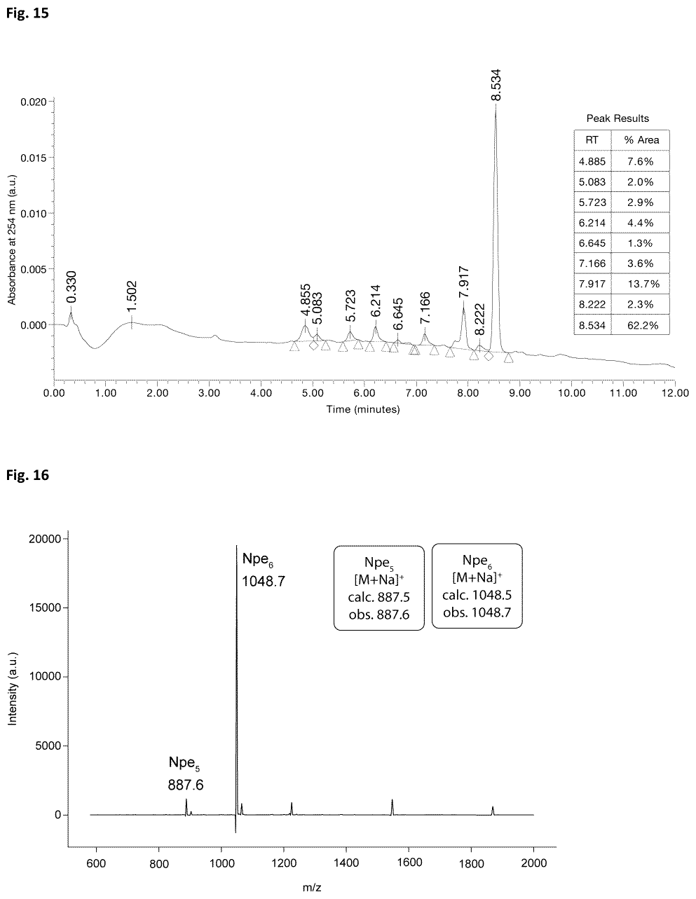

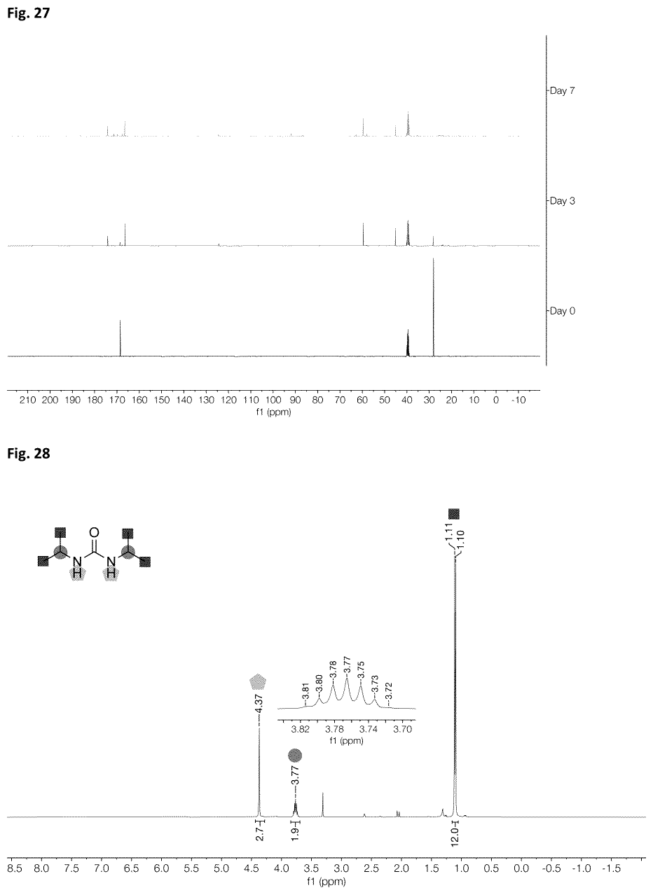

Therefore, an alternative green method using DMSO was designed to limit early termination of the peptoid chains and improve crude purities from the GVL method (Method 2 in Table 1). DMSO was selected as it was used for the displacement step in the initial reports of peptoid synthesis but was eventually replaced by NMP.¹¹ However, this method was unsuccessful in the synthesis of the 6-mer, due to the absence of the expected peak in the UHPLC and MALDI-TOF analyses (FIG. 3A, FIGS. 11-13). The shorter chain lengths that were observed with the GVL protocol by UHPLC (5-mer and 4-mer) were also not present with the DMSO method. Mass analysis revealed the presence of a 3-mer in the crude (observed=565.3, calculated=565.3), which we were unable to assign to a peak in the UHPLC due to the presence of many overlapping peaks (FIGS. 11-13). Without wishing to be bound to theory, the lack of higher chain lengths attained using this method was hypothesized to be the result of a reaction between bromoacetic acid (BrAc) and DMSO. Over the period of the synthesis, the 0.8M BrAc solution in DMSO changed from colourless to a bright yellow. A nuclear magnetic resonance (NMR) spectroscopy study of BrAc in DMSO-d6 over 7 days revealed complete conversion of bromoacetic acid into unreactive by-products (FIGS. 26-27). The acylation step was confirmed to be the cause of the incomplete reaction as changing the acylation solvent from DMSO to DMF while keeping all other steps in DMSO (Method 3 in Table 1) yielded the 6-mer with much improved purity from Method 1 and 2 (FIG. 3A). While the synthesis of the 6-mer was confirmed by UHPLC and MALDI-TOF (FIGS. 14-16), there was a loss in purity from the DMF/NMP method due to the presence of 5-mers in the crude product.

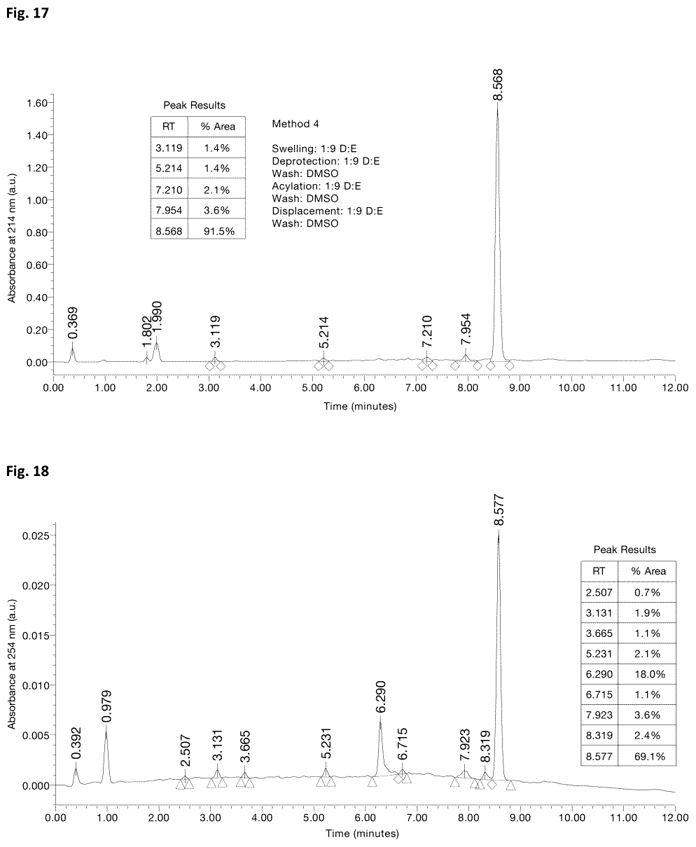

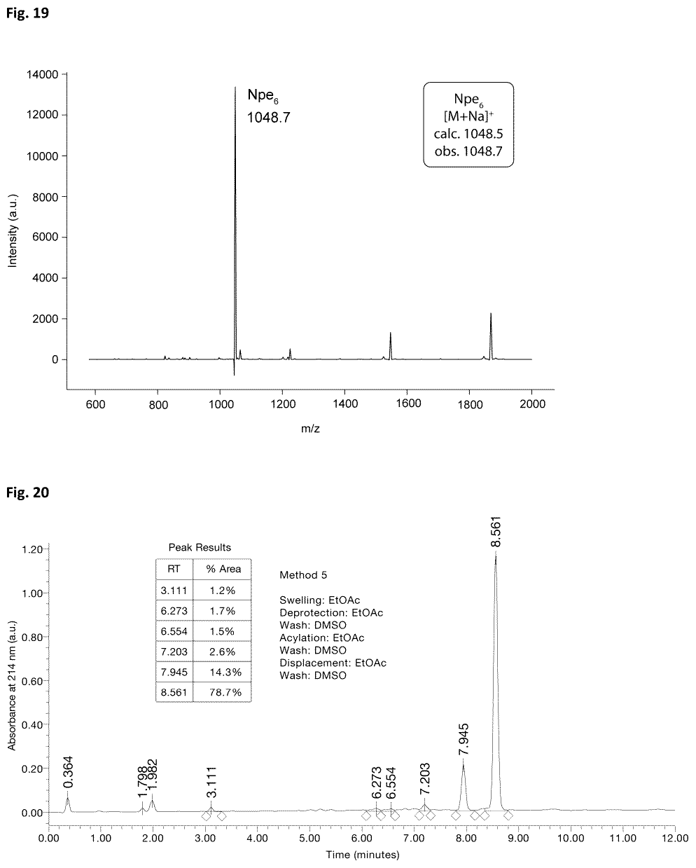

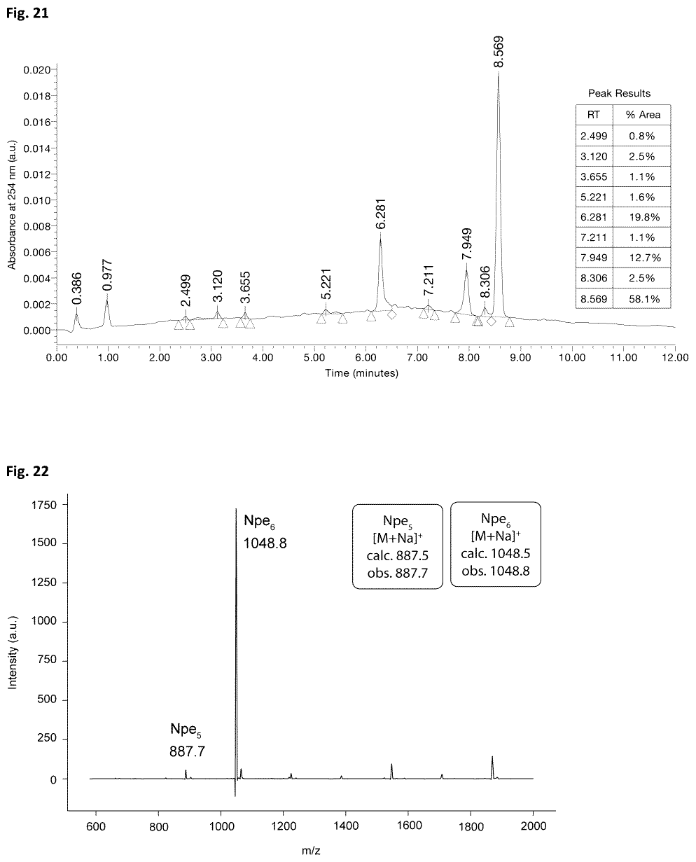

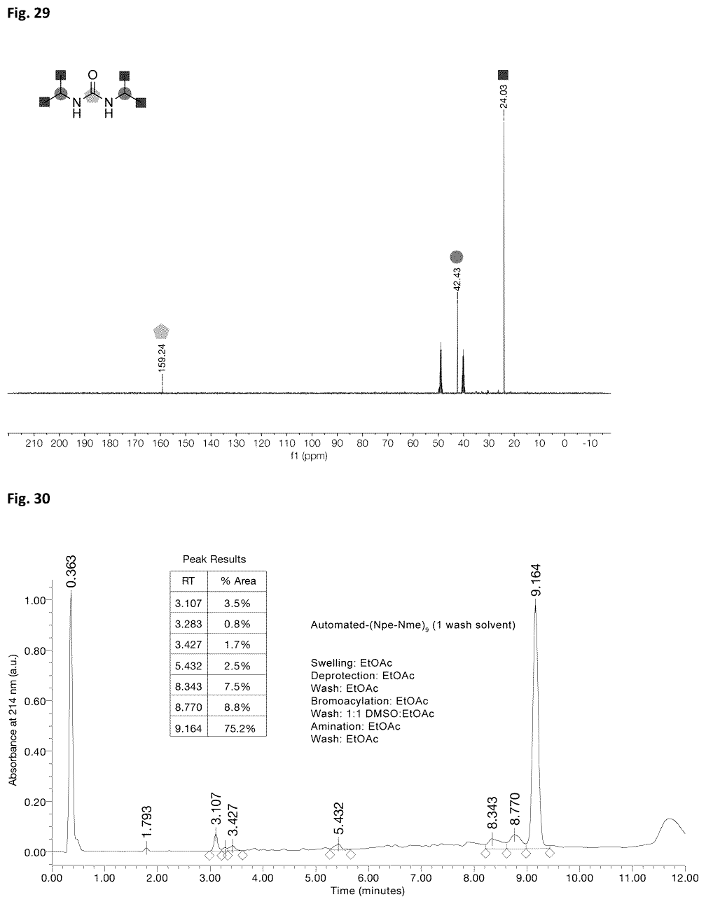

Using a solvent mixture with improved swelling capacity from that of DMSO could allow for more efficient reactions and increased purity, leading to the investigation of 1:9 DMSO: EtOAc (1:9 D: E) for green peptoid synthesis (Method 4 in Table 1). This mixture was initially attempted for all steps, but it was determined that the by-product formed during the acylation step was unable to be solubilized with 1:9 D: E. This was addressed by re-introducing DMSO for all washing steps. NMR analysis of the by-product revealed its identity as 1,3-diisopropylurea, formed during the reaction of N, N′-diisopropylcarbodiimide (DIC) (Scheme S2, FIGS. 28-29). The purity of the crude 6-mer was very similar to the traditional DMF/NMP method with minimal 5-mer observed by MALDI-TOF and UHPLC (FIG. 3A, FIGS. 17-19), suggesting that resin swelling plays an important role in reaction efficiency. However, it is more user-friendly for a greener peptoid synthesis protocol to use a single solvent than a mixture of solvents. Method 5 (Table 1) used EtOAc for the swelling, deprotection, and reaction steps (e.g. acylation and displacement) with DMSO as the acylation washing solvent to solubilize the 1,3-diisopropylurea by-product formed during acylation. The purity of the 6-mer decreased with the switch from 1:9 D: E to EtOAc. The increase in the presence of the 5-mer with Method 5, as observed by UHPLC and MALDI-TOF, was likely due to the lower swelling efficiency of EtOAc (FIG. 2-3, FIGS. 20-22).

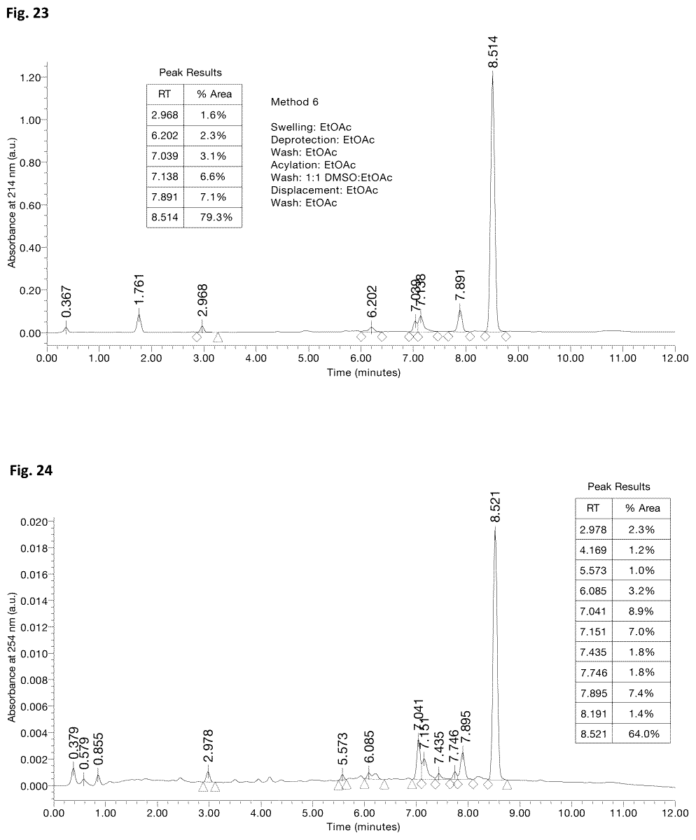

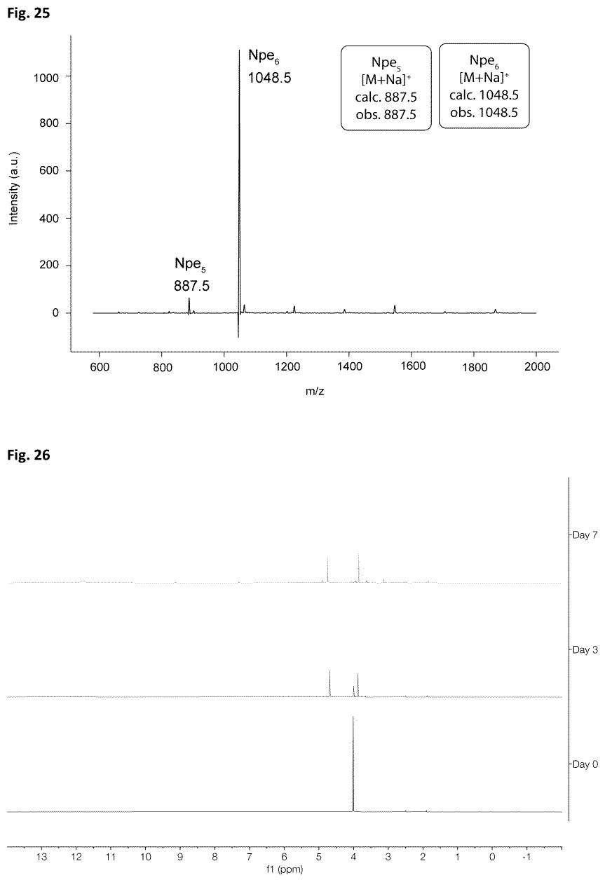

The use of DMSO was further reduced by replacing all washes with EtOAc except the acylation wash, which required 1:1 DMSO: EtOAc (1:1 D: E) due to the solubility of the 1,3-diisopropylurea by-product (Method 6 in Table 1). This method yielded a 6-mer with the same purity as Method 5, which used DMSO for all washes (FIG. 3A, 3C, FIGS. 23-25). The non-acetylated Npe6 peptoids synthesized using the DMF/NMP method and Method 6) were purified using recycling gel permeation chromatography (GPC) in chloroform. The pure 6-mer was obtained in the same yield between the two methods (Table 3). Peptoids are commonly purified using reverse-phase HPLC, which separates the crude mixture on the basis of polarity. Yields of 30-60% are commonly reported for pure fractions of peptoids obtained using reverse-phase high-performance liquid chromatography (HPLC). 20,40,41 The recycling GPC separates the crude mixture on the basis of size, yet adsorption to the packing material can occur as the peptoid is passed over it multiple times, reducing the final yields. However, the yield of Npe6 following purification by recycling GPC still fell within the range typically obtained using reverse-phase HPLC.

TABLE 3
Crude UHPLC purities of the acetylated 6-mer peptoid, Npe₆ (based
on area % of peak at ~8.5 min) and yields of non-acetylated 6-mer
peptoid, Npe₆ following purification by recycling GPC.

| | DMF/NMP | Method 1 | Method 2 | Method 3 | Method 4 | Method 5 | Method 6 |
|---|---|---|---|---|---|---|---|
| Crude | 95% | 56% | 0% | 80% | 92% | 79% | 79% |
| purity | | | | | | | |
| Yield^{a} | 51% | —^{b} | —^{b} | —^{b} | —^{b} | —^{b} | 51% |

^{a}Lower yields are due to the nature of the purification by recycling GPC.
^{b}Yields were only determined for the standard and greenest conditions.

### Example 5: Automated Solid-Phase Submonomer Synthesis of 6-Mer and 18-Mer Polypeptoids with Green Solvents

The manual synthesis of Npe6, was adapted for use with an automated synthesizer for the green synthesis of peptoids with higher molecular weights. The protocol used on the Gyros Protein Technologies PurePep® Chorus® is similar to the manual solid-phase submonomer peptoid synthetic protocols disclosed in Example 3 and 4. The automated synthesis protocol follows the manual protocol closely but increases the number of washes from 3 to 5 and the displacement time from 30 min to 60 min (Tables 4 and 5). The procedures are described in Tables 4 and 5 when using 1:1 DMSO: EtOAc for all washes (Table 4) or only for the acylation wash (Table 5). 100 mg of Rink amide-MBHA resin (0.62 mmol/g loading) was added to a 10 mL solid-phase peptide synthesis fritted reaction vessel. Cleavage and acetylation procedures were performed as stated in Example 3 and 4 for the manual solid-phase submonomer peptoid synthesis.

In addition to Npe6, the synthesis of an alternating 18mer with a new hydrophilic monomer was automated, N-(2-methoxyethyl) glycine (Nme) (Schemes S3-4). This side chain has chemical resemblance to poly (ethylene glycol) (PEG), a ubiquitous polymer used in several applications, such as antifouling surfaces.⁴²

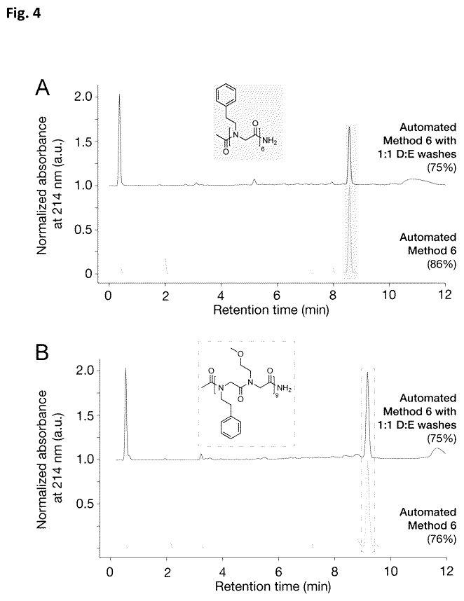

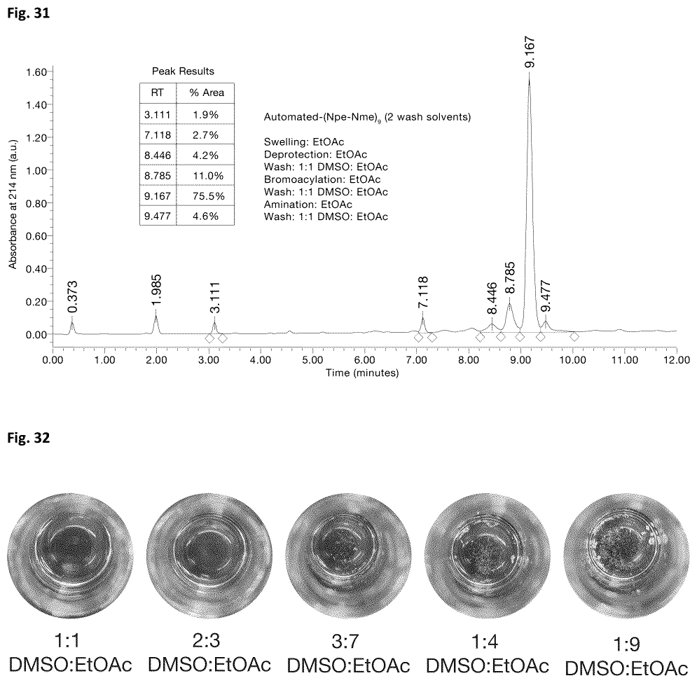

The automated synthesis of Npe6 using Method 6 requires the synthesizer to have the capacity to deliver two different washing solvents, which is not necessarily accessible for all automated synthesizers (Table 5). Therefore, a synthesizer with only one wash solvent was also programmed and tested, demonstrating that the the green or greener protocols described herein could be adapted to synthesizers with space for only a single wash solvent (Table 4). The 6-mer was synthesized successfully with a higher purity by UHPLC than obtained using the manual protocol (FIG. 4A bottom). It is hypothesized that the automated synthesizer is better sealed during mixing than the manual synthesizer, reducing EtOAc evaporation and allowing the resin to remain swollen for better reagent penetration. The single wash protocol also yielded the desired 6-mer with comparable purity to Method 6 (FIG. 4A top). Both methods were applied to the synthesis of the 18-mer which were successful by UHPLC and MALDI-TOF (FIG. 4B, FIGS. 30 and 31). This further supports the versatility of green peptoid synthesis, as it is compatible with protocols for automated peptoid synthesizers and is not limited by the desired peptoid chain length. Although the method described in Tables 4 and 5 uses dichloromethane (DCM) to de-swell the resin, it was found that this step is not necessary and can be skipped with no effect on final yields or purities.

TABLE 4
Green solid-phase submonomer peptoid synthesis method for an automated
peptide synthesizer using 1:1 DMSO:EtOAc for all washes.

| | | | Reaction | | | | |
|---|---|---|---|---|---|---|---|
| | | | time | Volume | Mixing | | |
| Step | Operation | Reagent | (h:m:s) | (mL) | conditions | Drain | Repetition |
| Resin | Swell | 1:1 | 00:00:30 | 3.00 | N₂ mixing | Yes | 1 |
| swelling + | Resin | DMSO:EtOAc | | | | | |
| deprotection | Swell | 1:1 | 00:10:00 | 3.00 | N₂ mixing, | Yes | 1 |
| | Resin | DMSO:EtOAc | | | shaking 350 | | |
| | | | | | RPM | | |
| | Fmoc | 20% 4- | 00:00:30 | 2.00 | N₂ mixing, | Yes | 1 |
| | deprotection | methylpiperidine | | | shaking 350 | | |
| | | in EtOAc | | | RPM, 35° C. | | |
| | Fmoc | 20% 4- | 00:12:00 | 2.00 | N₂ mixing, | Yes | 1 |
| | deprotection | methylpiperidine | | | shaking 350 | | |
| | | in EtOAc | | | RPM, 35° C. | | |
| | Wash | 1:1 | 00:00:30 | 3.00 | N₂ mixing, | Yes | 5 |
| | | DMSO:EtOAc | | | shaking 350 | | |
| | | | | | RPM | | |
| Peptoid | Acylation | 0.8M BrAc in | 00:00:30 | 1.00 | — | No | 1 |
| coupling | | EtOAc | | | | | |
| cycle | Acylation | 0.8M DIC in | 00:30:00 | 1.00 | N₂ mixing, | Yes | 1 |
| | | EtOAc | | | shaking 350 | | |
| | | | | | RPM, 35° C. | | |
| | Wash | 1:1 | 00:00:30 | 3.00 | N₂ mixing | Yes | 5 |
| | | DMSO:EtOAc | | | | | |
| | Displacement | 1M amine in | 01:00:00 | 2.00 | N₂ mixing, | Yes | 1 |
| | | EtOAc | | | shaking 350 | | |
| | | | | | RPM, 35° C. | | |
| | Wash | 1:1 | 00:00:30 | 3.00 | N₂ mixing | Yes | 5 |
| | | DMSO:EtOAc | | | | | |
| Resin | Wash | DCM | 00:00:30 | 3.00 | N₂ mixing | Yes | 5 |
| deswelling + | Drain to dry | — | 1:00:00 | — | — | Yes | 1 |
| drying | resin | | | | | | |

TABLE 5
Green solid-phase submonomer peptoid synthesis method for automated peptide synthesizer
using EtOAc for all washes except after acylation (1:1 DMSO:EtOAc).

| | | | Reaction | | | | |
|---|---|---|---|---|---|---|---|
| | | | time | Volume | Mixing | | |
| Step | Operation | Reagent | (h:m:s) | (mL) | conditions | Drain | Repetition |
| Resin | Swell | EtOAc | 00:00:30 | 3.00 | N₂ mixing | Yes | 1 |
| swelling + | Resin | | | | | | |
| deprotection | Swell | EtOAc | 00:10:00 | 3.00 | N₂ mixing, | Yes | 1 |
| | Resin | | | | shaking 350 | | |
| | | | | | RPM | | |
| | Fmoc | 20% 4- | 00:00:30 | 2.00 | N₂ mixing, | Yes | 1 |
| | deprotection | methylpiperidine | | | shaking 350 | | |
| | | in EtOAc | | | RPM, 35° C. | | |
| | Fmoc | 20% 4- | 00:12:00 | 2.00 | N₂ mixing, | Yes | 1 |
| | deprotection | methylpiperidine | | | shaking 350 | | |
| | | in EtOAc | | | RPM, 35° C. | | |
| | Wash | EtOAc | 00:00:30 | 3.00 | N₂ mixing, | Yes | 5 |
| | | | | | shaking 350 | | |
| | | | | | RPM | | |
| Peptoid | Acylation | 0.8M BrAc in | 00:00:30 | 1.00 | — | No | 1 |
| coupling | | EtOAc | | | | | |
| cycle | Acylation | 0.8M DIC in | 00:30:00 | 1.00 | N₂ mixing, | Yes | 1 |
| | | EtOAc | | | shaking 350 | | |
| | | | | | RPM, 35° C. | | |
| | Wash | 1:1 | 00:00:30 | 3.00 | N₂ mixing | Yes | 5 |
| | | DMSO:EtOAc | | | | | |
| | Displacement | 1M amine in | 01:00:00 | 2.00 | N₂ mixing, | Yes | 1 |
| | | EtOAc | | | shaking 350 | | |
| | | | | | RPM, 35° C. | | |
| | Wash | EtOAc | 00:00:30 | 3.00 | N₂ mixing | Yes | 5 |
| Resin | Wash | DCM | 00:00:30 | 3.00 | N₂ mixing | Yes | 5 |
| deswelling + | Drain to dry | — | 1:00:00 | — | — | Yes | 1 |
| drying | resin | | | | | | |

TABLE 6
Crude UHPLC purities of the acetylated 6-mer peptoid, Npe₆ (based on area
% of peak at ~8.5 min) and acetylated 18-mer peptoid, (Npe-Nme)₉ synthesized
using green automated solid-phase peptoid synthetic methods.

| | | Npe₆ | | (Npe-Nme)₉ |
|---|---|---|---|---|
| | | 2 wash solvents | | 2 wash solvents |
| | | (EtOAc for all | | (EtOAc for all |
| | | washes except | | washes except |
| | Npe₆ | acylation wash | (Npe-Nme)₉ | acylation wash |
| | wash solvent | is 1:1 | 1 wash solvent | is 1:1 |
| | (1:1 DMSO:EtOAc) | DMSO:EtOAc) | (1:1 DMSO:EtOAc) | DMSO:EtOAc) |
| Crude purity | 75% | 86% | 75% | 76% |

### Example 6

The 6-mer peptoid was synthesized according to Examples 1 and 3 using the following method. The acylation solvent was EtOAc, the acylation wash solvent was 1:1 DMSO: EtOAc, the displacement solvent was EtOAc, and the displacement wash solvent was 1:1 DMSO: EtOAc. The method worked to produce the 6-mer. The method produced the expected products with a comparable purity to the automatic synthesized 6-mer.

### Example 7

Different DMSO: EtOAc ratios for the acylation solvent wash were tested. The solubility of the 1,3-diisopropylurea by-product produced during the acylation step of solid-phase peptoid synthesis was investigated in different ratios of DMSO and EtOAc (FIG. 32). A 10 mg/mL solution of the 1,3-diisopropylurea by-product was prepared in 1:1 DMSO: EtOAc, 2:3 DMSO: EtOAc, 3:7 DMSO: EtOAc, 1:4 DMSO: EtOAc, and 1:9 DMSO: EtOAc. The by-product was soluble in the 1:1 DMSO: EtOAc solution and 2:3 DMSO: EtOAc solution but was insoluble in the 3:7 DMSO: EtOAc, 1:4 DMSO: EtOAc, and 1:9 DMSO: EtOAc solutions. The 1,3-diisopropylurea by-product was soluble in wash solvents with a DMSO concentration greater than 30% v/v.

### Schemes:

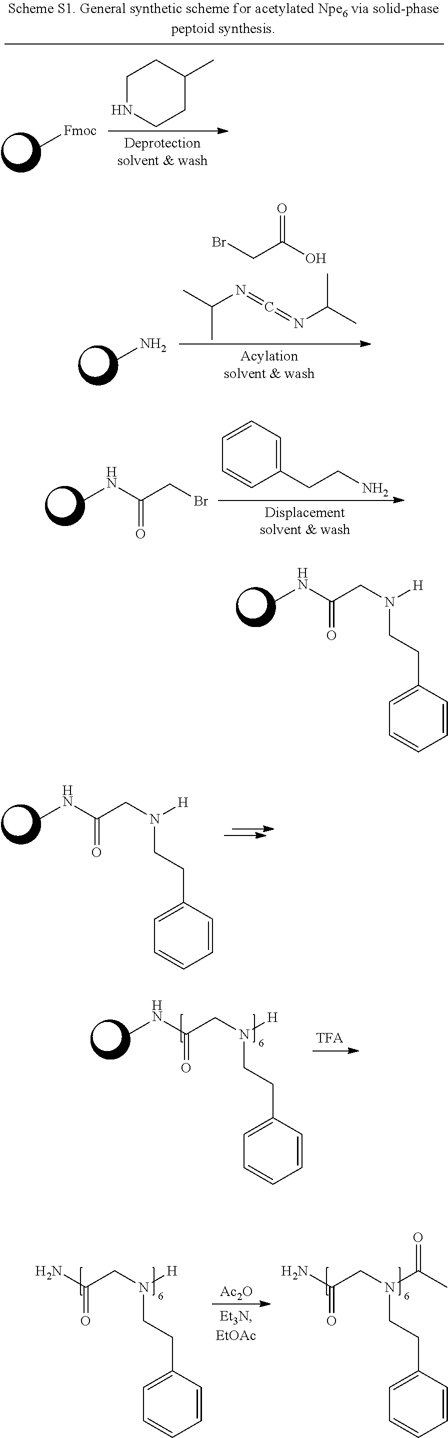

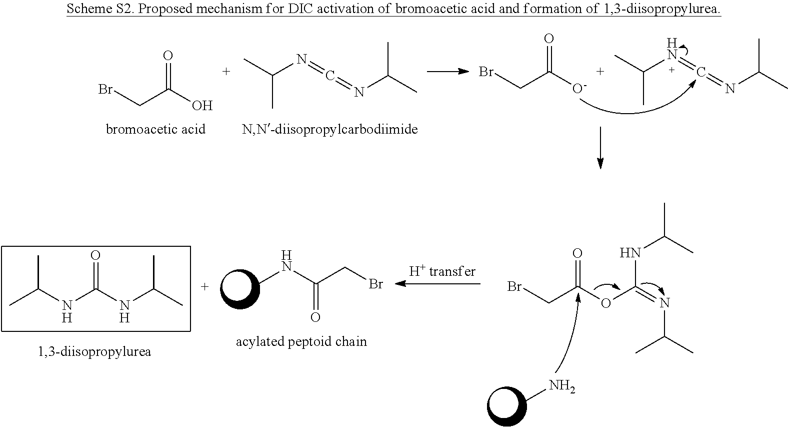

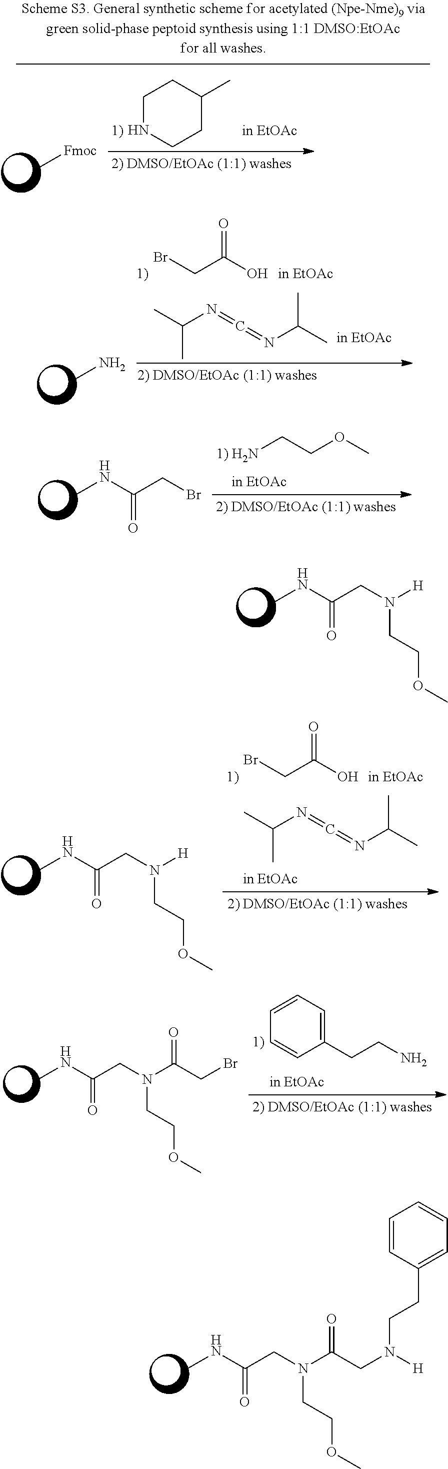

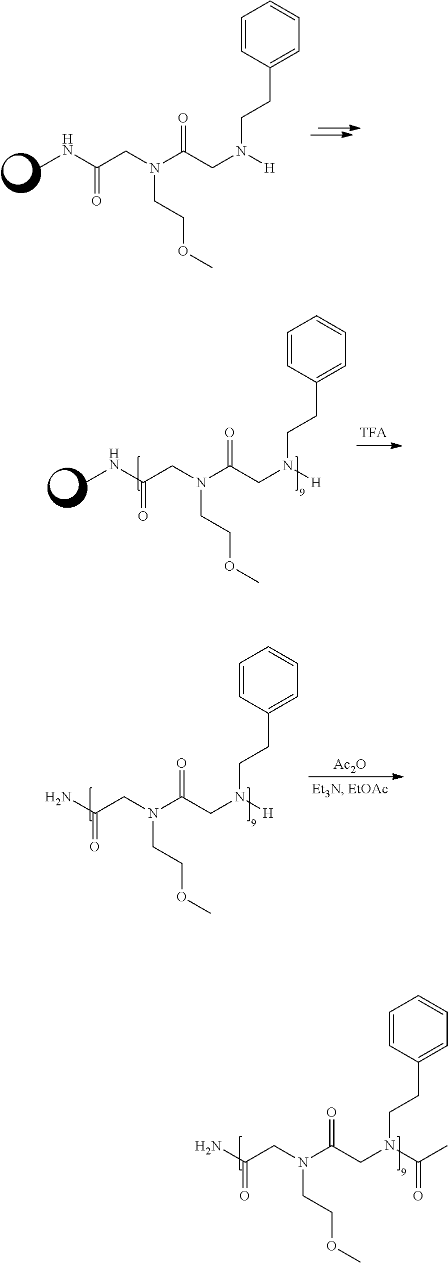

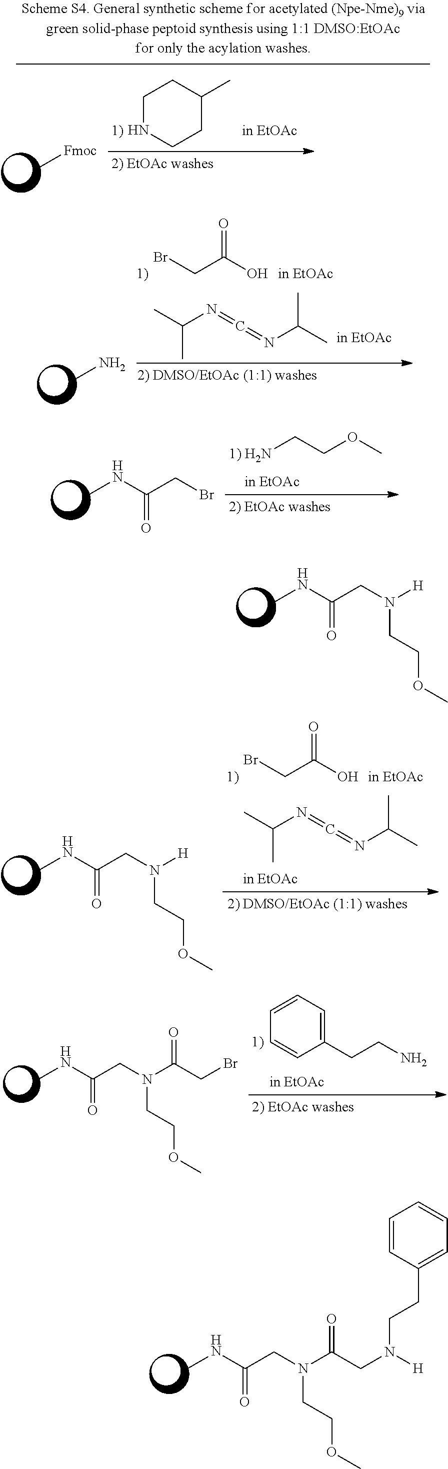

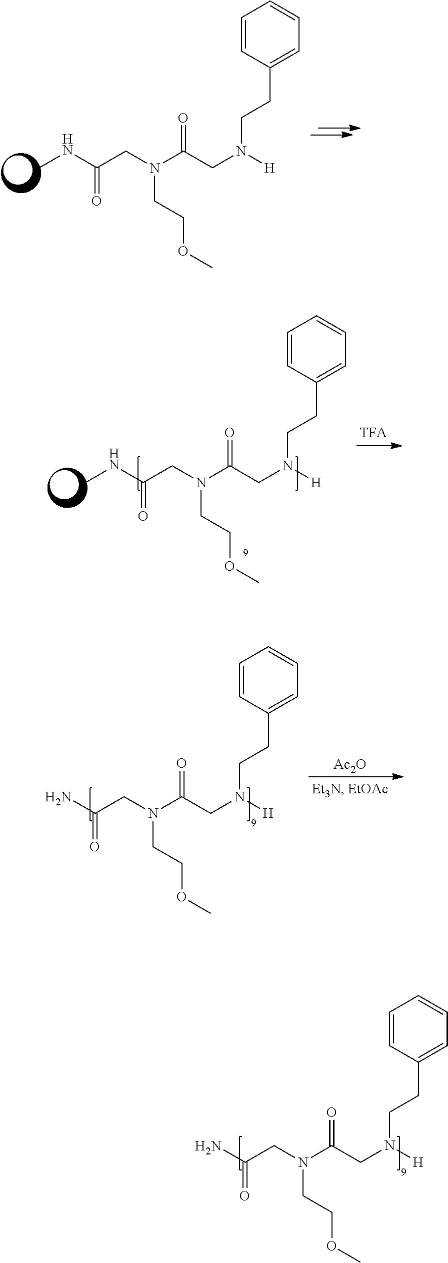

### Example 8

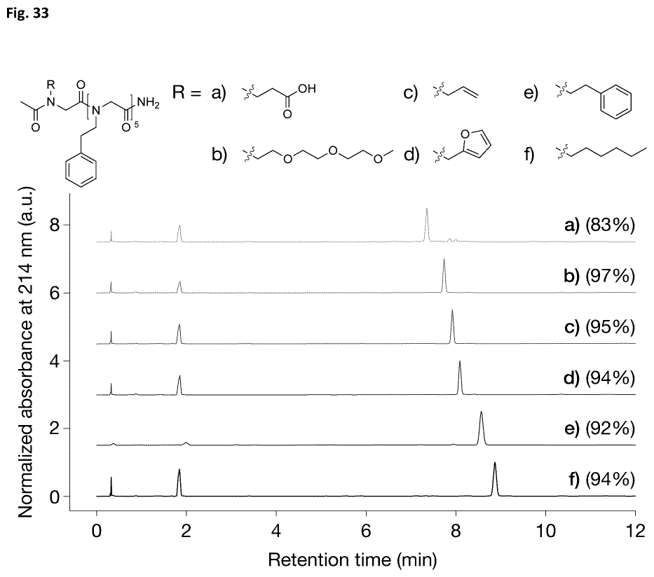

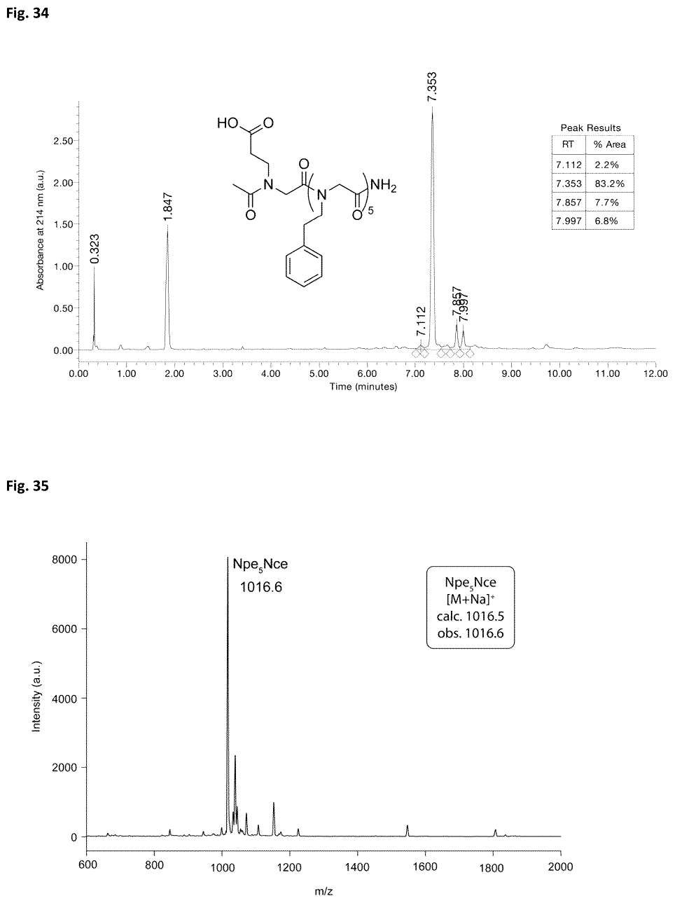

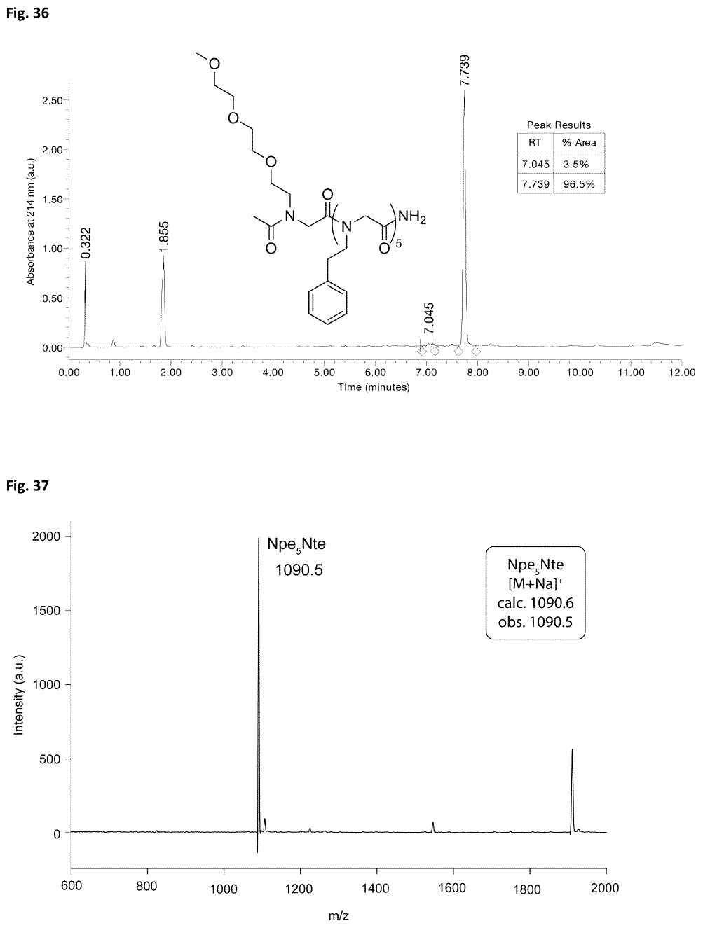

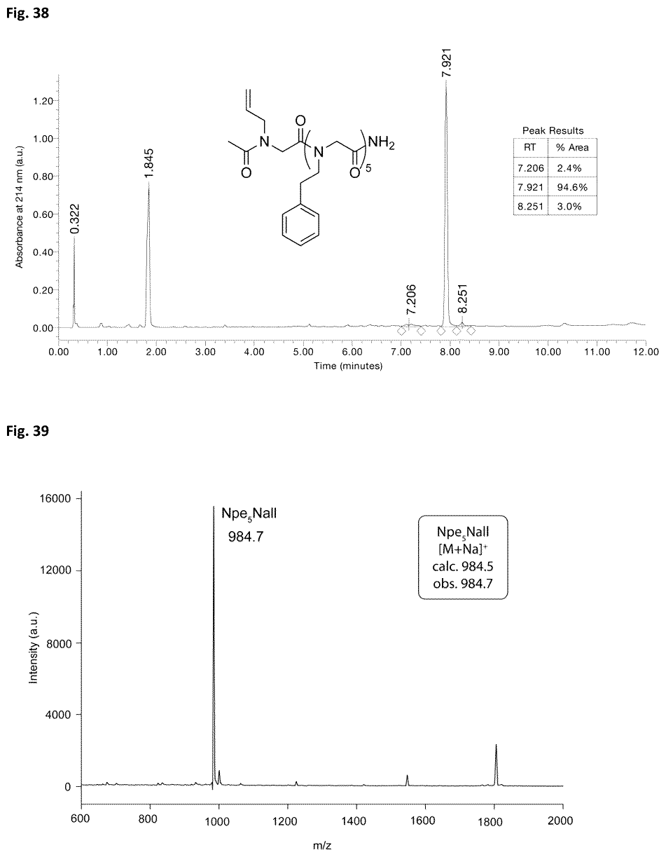

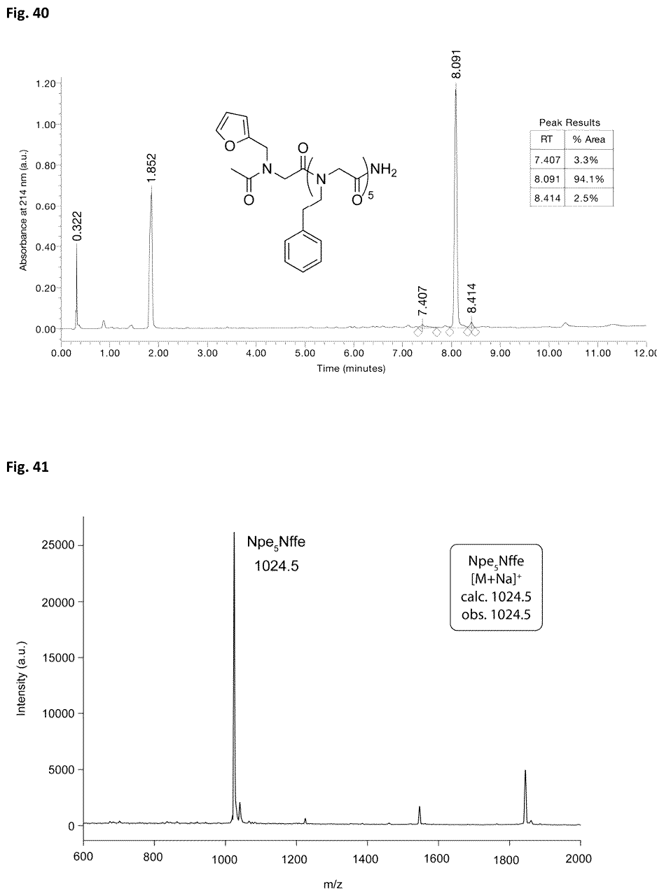

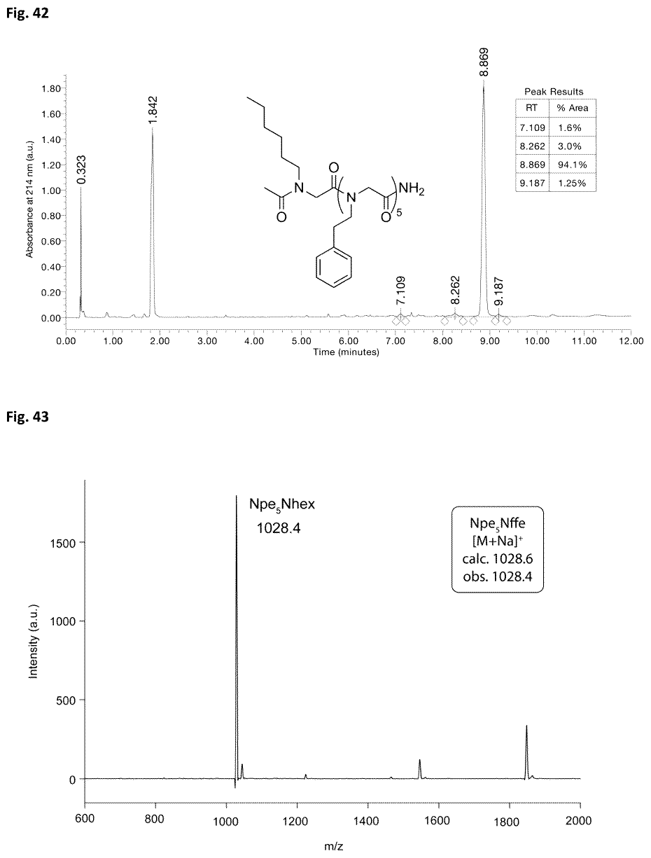

A diverse array of amines can be incorporated as side chains. The 6-mer peptoid was synthesized according to Examples 1 and 3 using the following method. The acylation solvent, the displacement solvent, the displacement wash solvent, the deprotection solvent and the deprotection wash solvent was 1:9 DMSO: EtOAc, and the acylation wash solvent was 1:1 DMSO: EtOAc. A diverse array of amines can be incorporated into the side chains. In this Example, amines with protecting groups (FIG. 33a), increased hydrophilicity (FIG. 33b), capacity for post-functionalization (FIG. 33c), heterocyclic functional groups (FIG. 33d), and increased hydrophobicity (FIG. 33f) were dissolved in 1:9 D: E and used in the submonomer synthesis. The purities of the synthesized peptoids were equivalent with what is observed in the traditional protocol with DMF/NMP (FIGS. 3, 33, 34-43). This example in no way limits the types of amines that can be used. Any number of repeating units may be synthesized with any type of primary amine.

### REFERENCES

1. Chen, X.; Wu, J.; Luo, Y.; Liang, X.; Supnet, C.; Kim, M. W.; Lotz, G. P.; Yang, G.; Muchowski, P. J.; Kodadek, T.; Bezprozvanny, I. Expanded Polyglutamine-Binding Peptoid as a Novel Therapeutic Agent for Treatment of Huntington's Disease. Chem Bio/2011, 18 (9), 1113-1125.
2. Bremner, J. B.; Keller, P. A.; Pyne, S. G.; Boyle, T. P.; Brkic, Z.; David, D. M.; Garas, A.; Morgan, J.; Robertson, M.; Somphol, K.; Miller, M. H.; Howe, A. S.; Ambrose, P.; Bhavnani, S.; Fritsche, T. R.; Biedenbach, D. J.; Jones, R. N.; Buckheit, R. W.; Watson, K. M.; Baylis, D.; Coates, J. A.; Deadman, J.; Jeevarajah, D.; McCracken, A.; Rhodes, D. I. Binaphthyl-Based Dicationic Peptoids with Therapeutic Potential. Angew Chem Int Ed 2010, 122 (3), 547-550.
3. Spicer, S. K.; Subramani, A.; Aguila, A. L.; Green, R. M.; McClelland, E. E.; Bicker, K. L. Toward a Clinical Antifungal Peptoid: Investigations into the Therapeutic Potential of AEC5. Biopolymers 2019, 110 (6), e23276.
4. Huang, M. L37erehre, D.; Jiang, Q.; Hu, C.; Kirshenbaum, K.; Ward, M. D. Biomimetic Peptoid Oligomers as Dual-Action Antifreeze Agents. Proc Natl Acad Sci U S A 2012, 109 (49), 19922-19927.
5. Zhu, L.; Zhao, Z.; Cheng, P.; He, Z.; Cheng, Z.; Peng, J.; Wang, H.; Wang, C.; Yang, Y.; Hu, Z. Antibody-Mimetic Peptoid Nanosheet for Label-Free Serum-Based Diagnosis of Alzheimer's Disease. Adv Mater 2017, 29 (30), 1700057.
6. Murray, D. J.; Kim, J. H.; Grzincic, E. M.; Kim, S. C.; Abate, A. R.; Zuckermann, R. N. Uniform, Large-Area, Highly Ordered Peptoid Monolayer and Bilayer Films for Sensing Applications. Langmuir 2019, 35 (42), 13671-13680.
7. Xie, J.; Zhou, M.; Qian, Y.; Cong, Z.; Chen, S.; Zhang, W.; Jiang, W.; Dai, C.; Shao, N.; Ji, Z.; Zou, J.; Xiao, X.; Liu, L.; Chen, M.; Li, J.; Liu, R. Addressing MRSA Infection and Antibacterial Resistance with Peptoid Polymers. Nat Commun 2021, 12 (1), 1-13.
8. Zhu, L.; Zhao, Z.; Cheng, P.; He, Z.; Cheng, Z.; Peng, J.; Wang, H.; Wang, C.; Yang, Y.; Hu, Z. Antibody-Mimetic Peptoid Nanosheet for Label-Free Serum-Based Diagnosis of Alzheimer's Disease. Adv Mater 2017, 29 (30).
9. Gao, H.; Wang, J.; Liu, J.; Ye, S.; Meng, X.; Song, S.; Wang, C.; Yu, X.; Zhu, L.; Wang, H.; Lei, S.; Yang, Y. Peptoid Nanosheet-Based Sensing System for the Diagnosis and Surveillance of Amnestic Mild Cognitive Impairment and Alzheimer's Disease. ACS Chem Neurosci 2021, 12 (22), 4257-4264.
10. Gao, H.; Liu, M.; Zhao, Z.; Yang, C.; Zhu, L.; Cai, Y.; Yang, Y.; Hu, Z. Diagnosis of Mild Cognitive Impairment and Alzheimer's Disease by the Plasma and Serum Amyloid-Beta 42 Assay through Highly Sensitive Peptoid Nanosheet Sensor. ACS Appl Mater Interfaces 2020, 12 (8), 9693-9700.
11. Zuckermann, R. N.; Kerr, J. M.; Moosf, W. H.; Kent, S. B. H. Efficient Method for the Preparation of Peptoids [Oligo (N-Substituted Glycines)] by Submonomer Solid-Phase Synthesis. J Am Chem Soc 1992, 114 (26), 10646-10647.
12. Connolly, M. D.; Xuan, S.; Molchanova, N.; Zuckermann, R. N. Submonomer Synthesis of Sequence Defined Peptoids with Diverse Side-Chains. Methods Enzymol 2021, 656, 241-270.
13. Clapperton, A. M.; Babi, J.; Tran, H. A Field Guide to Optimizing Peptoid Synthesis. ACS Polym Au 2022, 2, 417-429.
14. Battigelli, A.; Kim, J. H.; Dehigaspitiya, D. C.; Proulx, C.; Robertson, E. J.; Murray, D. J.; Rad, B.; Kirshenbaum, K.; Zuckermann, R. N. Glycosylated Peptoid Nanosheets as a Multivalent Scaffold for Protein Recognition. ACS Nano 2018, 12 (3), 2455-2465.
15. Caumes, C.; Roy, O.; Faure, S.; Taillefumier, C. The Click Triazolium Peptoid Side Chain: A Strong Cis-Amide Inducer Enabling Chemical Diversity. J Am Chem Soc 2012, 134 (23), 9553-9556.
16. Darapaneni, C. M.; Kaniraj, P. J.; Maayan, G. Water Soluble Hydrophobic Peptoids via a Minor Backbone Modification. Org Biomol Chem 2018, 16 (9), 1480-1488.
17. Burkoth, T. S.; Fafarman, A. T.; Charych, D. H.; Connolly, M. D.; Zuckermann, R. N. Incorporation of Unprotected Heterocyclic Side Chains into Peptoid Oligomers via Solid-Phase Submonomer Synthesis. J Am Chem Soc 2003, 125 (29), 8841-8845.
18. Proulx, C.; Yoo, S.; Connolly, M. D.; Zuckermann, R. N. Accelerated Submonomer Solid-Phase Synthesis of Peptoids Incorporating Multiple Substituted N-Aryl Glycine Monomers. J Org Chem 2015, 80 (21), 10490-10497.
19. Zuckermann, R. N. Peptoid Origins. Biopolymers 2011, 96 (5), 545-555.
20. Tran, H.; Gael, S. L.; Connolly, M. D.; Zuckermann, R. N. Solid-Phase Submonomer Synthesis of Peptoid Polymers and Their Self-Assembly into Highly-Ordered Nanosheets. J Vis Exp 2011, No. 57.
21 Sherwood, J.; Farmer, T. J.; Clark, J. H. Catalyst: Possible Consequences of the N-Methyl Pyrrolidone REACH Restriction. Chem 2018, 4 (9), 2010-2012.
22. Borchert, F.; Beronius, A.; Ågerstrand, M. Characterisation and Analysis of Key Studies Used to Restrict Substances under REACH. Environ Sci Eur 2022, 34 (1), 1-15.
23. Bryan, M. C.; Dunn, P. J.; Entwistle, D.; Gallou, F.; Koenig, S. G.; Hayler, J. D.; Hickey, M. R.; Hughes, S.; Kopach, M. E.; Moine, G.; Richardson, P.; Roschangar, F.; Steven, A.; Weiberth, F. J. Key Green Chemistry Research Areas from a Pharmaceutical Manufacturers' Perspective Revisited. Green Chem 2018, 20 (22), 5082-5103.
24 Substance Informati-n-ECHA https://echa.europa.eu/substance-information/-/substanceinfo/100.011.662 (accessed 2023 Feb. 14).
25. Substance Informati-n-ECHA https://echa.europa.eu/substance-information/-/substanceinfo/100.000.617 (accessed 2023 Feb. 14).
26. Jadhav, S.; Martin, V.; Egelund, P. H. G.; Johansson Castro, H.; Krüger, T.; Richner, F.; Thordal Le Quement, S.; Albericio, F.; Dettner, F.; Lechner, C.; Schönleber, R.; Pedersen, D. S. Replacing DMF in Solid-Phase Peptide Synthesis: Varying the Composition of Green Binary Solvent Mixtures as a Tool to Mitigate Common Side-Reactions. Green Chem 2021, 23 (9), 3312-3321.
27. Martin, V.; Jadhav, S.; Egelund, P. H. G.; Liffert, R.; Johansson Castro, H.; Krüger, T.; Haselmann, K. F.; Thordal Le Quement, S.; Albericio, F.; Dettner, F.; Lechner, C.; Schönleber, R.; Pedersen, D. S. Harnessing Polarity and Viscosity to Identify Green Binary Solvent Mixtures as Viable Alternatives to DMF in Solid-Phase Peptide Synthesis. Green Chem 2021, 23 (9), 3295-3311.
30. Lopez, J.; Pletscher, S.; Aemissegger, A.; Bucher, C.; Gallou, F. N-Butylpyrrolidinone as Alternative Solvent for Solid-Phase Peptide Synthesis. Org Process Res Dev 2018, 22 (4), 494-503.
31. Ferrazzano, L.; Corbisiero, D.; Martelli, G.; Tolomelli, A.; Viola, A.; Ricci, A.; Cabri, W. Green Solvent Mixtures for Solid-Phase Peptide Synthesis: A Dimethylformamide-Free Highly Efficient Synthesis of Pharmaceutical-Grade Peptides. ACS Sustain Chem Eng 2019, 7 (15), 12867-12877.
32. Nam, K. T.; Shelby, S. A.; Choi, P. H.; Marciel, A. B.; Chen, R.; Tan, L.; Chu, T. K.; Mesch, R. A.; Lee, B. C.; Connolly, M. D.; Kisielowski, C.; Zuckermann, R. N. Free-Floating Ultrathin Two-Dimensional Crystals from Sequence-Specific Peptoid Polymers. Nat Mater 2010, 9 (5), 454-460.
33. Sanii, B.; Kudirka, R.; Cho, A.; Venkateswaran, N.; Olivier, G. K.; Olson, A. M.; Tran, H.; Harada, R. M.; Tan, L.; Zuckermann, R. N. Shaken, Not Stirred: Collapsing a Peptoid Monolayer to Produce Free-Floating, Stable Nanosheets. J Am Chem Soc 2011, 133 (51), 20808-20815.
34. Robertson, E. J.; Proulx, C.; Su, J. K.; Garcia, R. L.; Yoo, S.; Nehls, E. M.; Connolly, M. D.; Taravati, L.; Zuckermann, R. N. Molecular Engineering of the Peptoid Nanosheet Hydrophobic Core. Langmuir 2016, 32 (45), 11946-11957.
40. Bolt, H. L.; Denny, P. W.; Cobb, S. L. An Efficient Method for the Synthesis of Peptoids with Mixed Lysine-Type/Arginine-Type Monomers and Evaluation of Their Anti-Leishmanial Activity. J Vis Exp 2016, 2016 (117).
41. Pratt, E. J.; Mancera-Andrade, E. I.; Bicker, K. L. Synthesis and Characterization of Derivatives of the Antifungal Peptoid RMG8-8. ACS Omega 2022, 7 (41), 36663-36671.
42. Statz, A. R.; Meagher, R. J.; Barron, A. E.; Messersmith, P. B. New Peptidomimetic Polymers for Antifouling Surfaces. J Am Chem Soc 2005, 127 (22), 7972-7973.
43. Culf, A. S.; Ouellette, R. J. Solid-Phase Synthesis of N-Substituted Glycine Oligomers (a-Peptoids) and Derivatives. Molecules 2010, 15, 5282-5335.

1. A method of synthesizing a peptoid, the method comprising
a) reacting a solid support comprising a terminal amine group, with a haloacetic acid (HA) in the presence of a HA activating agent in an acylation solvent, to obtain a solid support resin comprising an acylated amino group;
b) washing the solid support comprising the acylated amino group with an acylation wash solvent to obtain a washed solid support resin comprising the acylated amino group;
c) reacting the washed solid support comprising the acylated amino group with a primary amine in a displacement solvent, to obtain a washed solid support comprising an acylated resin-bound amide;
d) washing the solid support comprising the acylated resin-bound amide, with a displacement wash solvent, to obtain an attached peptoid monomer comprising an amine group;
e) repeating steps a)-d) at least once, where the amine group is the terminal amine group in step a) to obtain a peptoid;
wherein the acylation solvent, and the displacement solvent, are polar aprotic solvents that are non-reactive to secondary amines and optionally the acylation solvent provides for a resin swelling of at least 1.25, 1.3, 1.35, 1.4, 1.45 or 1.5 and/or the acylation wash solvent is able to solubilize 1,3-diisopropylurea; or
wherein the acylation solvent is selected from EtOAc, or a DMSO and EtOAc mixture, or
wherein the acylation wash solvent is selected from DMSO, EtOAc, or a mixture thereof, and
wherein when the acylation solvent is EtOAc, the acylation wash solvent is not EtOAc, and when the acylation wash solvent is DMSO, the acylation solvent is not DMSO.

2. The method of claim 1, wherein the haloacetic acid is selected from a bromoacetic acid, chloroacetic acid, or iodoacetic acid.

3.-4. (canceled)

5. The method of claim 1, wherein the acylation solvent is a DMSO and EtOAc mixture or a mixture comprising DMSO, optionally wherein the mixture is a ratio of DMSO: EtOAc from about 1:1 to 1:9 or wherein the DMSO is at least 10%, at least 20%, at least 30%, at least 40% or at least or up to 50% DMSO.

6.-8. (canceled)

9. The method of claim 1, wherein the acylation wash solvent is a DMSO and EtOAc mixture or a mixture comprising DMSO, optionally wherein the mixture is a ratio of DMSO: EtOAc wherein the DMSO is greater than 30% or 35%, or is at least 35% or 40%, optionally wherein the ratio is from about 4:6 to 1:1, optionally wherein the ratio is about 1:1 DMSO: EtOAc or wherein the DMSO is at least 40% or 50%.

10.-11. (canceled)

12. The method of claim 1, wherein the displacement solvent is or comprises DMSO or EtOAc.

13. The method of claim 1, wherein the displacement solvent is a DMSO and EtOAc mixture or a mixture comprising DMSO or a mixture comprising EtOAc, optionally wherein the mixture is a ratio of DMSO: EtOAc from about 10:1 to 1:9 or is a mixture comprising at least or about 10% DMSO.

14.-15. (canceled)

16. The method of claim 1, wherein the displacement wash solvent is or comprises DMSO or EtOAc.

17. The method of claim 1, wherein the displacement wash solvent is a DMSO and EtOAc mixture or a mixture comprising DMSO or a mixture comprising EtOAc, optionally wherein the displacement wash solvent comprises DMSO that is about or greater than 10%, 20%, 30% or 35%, or is or is at least 35% or 40%, optionally wherein the displacement wash solvent is a ratio of DMSO: EtOAc from about 1:9 to 1:1.

18. (canceled)

19. The method of claim 1, wherein the acylation wash solvent and the displacement wash solvent are the same solvent.

20. The method of claim 1, wherein the acylation solvent is EtOAc, the acylation wash solvent comprises a mixture of DMSO and EtOAc, the displacement solvent is or comprises EtOAc, and the displacement wash solvent is or comprises EtOAc.

21. The method of claim 1, wherein
the acylation solvent is a mixture of DMSO and EtOAc, the acylation wash solvent is DMSO, the displacement solvent is a mixture of DMSO and EtOAc, and the displacement wash solvent is DMSO;
the acylation solvent is about 1:9 DMSO: EtOAc, the acylation solvent wash is DMSO, the displacement solvent is about 1:9 DMSO: EtOAc, and the displacement wash solvent is DMSO;
the acylation solvent is EtOAc, the acylation wash solvent is DMSO, the displacement solvent is EtOAc, and the displacement wash solvent is DMSO;
the acylation solvent is EtOAc, the acylation solvent wash is about 1:1 DMSO: EtOAc, the displacement solvent is or comprises EtOAc, and the displacement wash solvent is about 1:1 DMSO: EtOAc;
the acylation solvent is EtOAc, the acylation wash solvent is about 1:1 DMSO: EtOAc, the displacement solvent is EtOAc, and the displacement wash solvent is EtOAc;
the acylation solvent is a mixture of DMSO and EtOAc, the acylation wash solvent is a mixture of DMSO and EtOAc, the displacement solvent is a mixture of DMSO and EtOAc, and the displacement wash solvent is a mixture of DMSO and EtOAc; or
the acylation solvent is about 1:9 DMSO: EtOAc, the acylation wash solvent is about 1:1 DMSO: EtOAc, the displacement solvent is about 1:9 DMSO: EtOAc, and the displacement wash solvent is about 1:9 DMSO: EtOAc.

22.-27. (canceled)

28. The method of claim 1, wherein the peptoid comprises a plurality of monomers of similar structure or wherein the peptoid comprises a plurality of monomers of different structures.

29.-30. (canceled)

31. The method of claim 1, wherein the peptoid is synthesized manually, or wherein the peptoid is synthesized using an automated peptoid synthesizer.

32. (canceled)

33. The method of claim 1, wherein the peptoid is separated from the solid support using a cleaving agent to obtain a crude peptoid.

34. The method of claim 1, wherein the solid support is a solid support resin, optionally selected from a Rink Amide 4-methylbenzhydrylamine (MBHA) resin, a Rink Amide resin, a Wang resin, or a 2-chlorotrityl chloride resin.

35. (canceled)

36. The method of claim 1, wherein prior to reacting a solid support comprising a terminal amine group, a protected terminal amine group is deprotected with a deprotection agent, optionally 4-methylpiperidine, in a deprotection solvent and washed with a deprotection wash solvent, to expose the terminal amine group.

37. The method of claim 1, wherein prior to deprotecting the terminal amine group, the solid support is a solid support resin and is solvated with a swelling solvent.

38. The method of claim 1, wherein the deprotection solvent is or comprises EtOAc or is or comprises DMSO.

39. The method of claim 36, wherein the deprotection solvent is a mixture of DMSO and EtOAC or a mixture comprising DMSO or a mixture comprising EtOAc, optionally wherein the ratio of DMSO: EtOAc is about 1:9 or the DMSO is or wherein the DMSO is at least 10%, at least 20%, at least 30%, at least 40% or at least or up to 50% DMSO.

40. (canceled)

41. The method of claim 36, wherein the deprotection wash solvent is or comprises DMSO or EtOAc or a mixture thereof.

42. The method of claim 36, wherein the deprotection wash solvent is a mixture of DMSO and EtOAc, optionally wherein the mixture is a ratio of DMSO: EtOAc from about 9:1 to about 1:9.

43. (canceled)

44. The method of claim 36, wherein
the deprotection solvent is about 1:9 DMSO: EtOAc, and the deprotection wash solvent is DMSO;
the deprotection solvent is EtOAc, and the deprotection wash solvent is DMSO;
the deprotection solvent is EtOAc, and the deprotection wash solvent is about 1:1 DMSO: EtOAc; or
the deprotection solvent is about 1:9 DMSO: EtOAc, and the deprotection wash solvent is about 1:9 DMSO: EtOAc.

45.-47. (canceled)
